(19) United States
(12) Patent Application Publication
(10) Pub. No.: US 2024/0010590 A1
(43) Pub. Date: Jan. 11, 2024

(54) Methods for preparing and purifying environmentally compatible detergents

(71) Applicant: TAKEDA PHARMACEUTICAL COMPANY LIMITED, Osaka (JP)

(72) Inventors: Jean-Baptiste FARCET, Vienna (AT); Otto KOSTNER, Vienna (AT)

(73) Assignee: TAKEDA PHARMACEUTICAL COMPANY LIMITED, Osaka (JP)

(21) Appl. No.: 18/019,640

(22) PCT Filed: Aug. 5, 2021
(86) PCT No.: PCT/IB2021/057190

§ 371 Date: Feb. 3, 2023

Related U.S. Application Data
(60) Provisional application No. 63/061,335, filed on Aug. 5, 2020.

(51) Int. Cl. C07C 41/09; C07C 41/16; C07C 41/38; C11D 1/72; C11D 3/43; C11D 3/48

(52) U.S. Cl. CPC C07C 41/09; C07C 41/16; C07C 41/38; C11D 1/721; C11D 3/43; C11D 3/48

(57) ABSTRACT

The invention provides methods for preparing and purifying compounds which can be used as environmentally compatible detergents, and compounds obtainable by such methods.

### FIELD OF THE INVENTION

The invention provides methods for preparing and purifying compounds which can be used as environmentally compatible detergents, and compounds obtainable by such methods.

### BACKGROUND

The use of biopharmaceutical drugs has continued to increase in importance as a method of treating many diseases, disorders, or conditions that affect an individual's health. Biopharmaceutical drugs are typically obtained by purification from a biological fluid or through recombinant production in host cells such as mammalian cell lines. However, in such biopharmaceutical production processes, viral contamination poses a significant problem. Viral contamination can be introduced to the biopharmaceutical production process by the biological fluids to be purified, or through the use of animal-derived products. In contrast to bacterial contamination, viral contamination is difficult to detect. However, if viral contamination goes unnoticed and infectious virus is incorporated into the formulation of the biopharmaceutical drug, it poses a significant health risk to patients. Therefore, viral inactivation is of paramount importance in biopharmaceutical production.

In many biopharmaceutical production processes, detergents are used for virus inactivation. Often, these detergents are combined with solvents in a so-called solvent/detergent (S/D) treatment. The detergent Triton X-100 (4-(1,1,3,3-tetramethylbutyl)phenol, ethoxylated) has been used for many years for S/D treatment of commercial products.

However, recent ecological studies have suggested that Triton X-100 and its degradation products can potentially behave as endocrine disrupters in aquatic organisms, raising environmental impact concerns (see “ECHA Support document for identification of 4-(1,1,3,3-tetramethylbutyl)phenol, ethoxylated as substances of very high concern because, due to their degradation to a substance of very high concern (4-(1,1,3,3-tetramethylbutyl)phenol) with endocrine disrupting properties, they cause probable serious effects to the environment which give rise to an equivalent level of concern to those of CMRs and PBTs/vPvBs”, adopted on 12 Dec. 2012). Consequently, alternative, environmentally compatible detergents for viral inactivation are required. WO 2019/086463 relates to such detergents for viral inactivation and to methods for their production. However, improved methods for the preparation and purification of these compounds are needed, and compounds of higher purity are desirable.

### DESCRIPTION OF THE INVENTION

The present invention meets the above-described needs and solves the above-mentioned problems in the art by providing the embodiments of the present invention.

The alleged toxicity of Triton-X100 purportedly arises from its phenol moiety, which has the ability to dock in certain endocrine receptors of marine life organisms. Consistently, based on in silico predictions of endocrine disruptor activity, for none of the non-phenolic polyoxyethylene ether detergents according to the present invention there was any prediction that they are active as endocrine disruptors.

The inventors have synthesized an environmentally compatible, non-phenolic polyoxyethylene ether detergent that efficiently inactivates lipid-enveloped viruses in S/D and single-detergent treatment. Hence, the inventors found that environmentally compatible, non-phenolic polyoxyethylene ether detergents can be used for inactivating lipid-enveloped viruses.

Furthermore, the inventors have developed improved methods for the preparation and purification of the detergents of the invention, as well as detergents obtainable by such methods having improved purity.

Accordingly, the present invention provides the preferred embodiments described below:
1. A method for preparing and purifying a compound of the following general Formula (XIX)

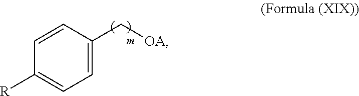

wherein m=1, R represents a hydrocarbon group having a linear chain of 2 to 12 carbon atoms and one or more methyl groups as substituents on said linear chain, and A represents a polyoxyethylene residue,
the method comprising the steps of
   (3) Reacting a compound of the following general Formula (XII)

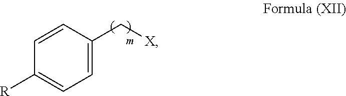

wherein m and R are as defined above and X represents a halogen atom or a hydroxyl group,
with a polyethylene glycol (PEG) of the general formula HO(CH₂CH₂O)ₙH, wherein n is an integer of n≥2, to produce a compound of said general formula (XIX) and a side product of the following general Formula (XIII)

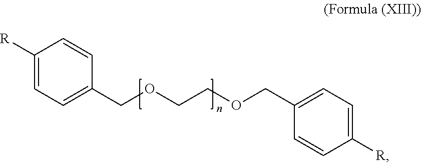

wherein R is as defined above and n is an integer of n≥2;
and
(4) Purifying the compound of said general Formula (XIX) by removing therefrom unreacted polyethylene glycol (PEG) and said side product.
2. The method of item 1, which further comprises the following step (2) prior to said step (3):
   (2) Converting a compound of the following general Formula (XIV),

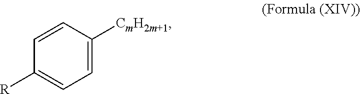

wherein m and R are as defined in item 1,
into a compound of the following general Formula (XII)

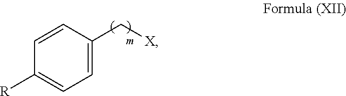

wherein m, R and X are as defined in item 1.
3. The method of item 2, which further comprises the following step (1) prior to said step (2):
   (1) Reacting toluene so as to obtain a compound of the following general Formula (XIV)

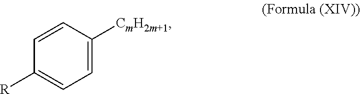

wherein m and R are as defined in item 1.
4. The method of any one of the preceding items, wherein in step (3), said polyethylene glycol (PEG) is used both as a reactant and as a solvent.
5. The method of item 3 or 4, wherein in step (1), toluene is reacted with diisobutylene or a compound R-X, wherein R is as defined in item 1 and X represents a halogen atom.
6. The method of any of items 2 to 5, wherein the converting in step (2) is a radical reaction using AIBN (azobis(isobutyronitrile)) as a radical initiator.
7. The method of any of items 1 to 6, wherein X is a chlorine, iodine or bromine atom.
8. The method of any of items 1 to 7, wherein X is a bromine atom.
9. The method of any of items 2 to 8, wherein the converting in step (2) uses N-bromosuccinimide (NBS) as a halogenating reagent.
10. The method of any one of the preceding items, wherein said step (4) comprises subjecting a solution of the compound of said general Formula (XIX) in a solvent selected from alkanes, ethers and esters and mixtures thereof to one or more aqueous washes to remove said unreacted polyethylene glycol (PEG) from the compound of said general Formula (XIX).
11. The method of item 10, wherein said solvent selected from alkanes, ethers and esters and mixtures thereof is an ester.
12. The method of item 11, wherein said solvent selected from alkanes, ethers and esters and mixtures thereof is ethyl acetate.
13. The method of any one of the preceding items, wherein the purifying of said compound of said general Formula (XIX) by removing therefrom said side product in step (4) comprises subjecting a solution of said compound of said general Formula (XIX) in a mixture comprising water and a water-miscible organic solvent to one or more washes with a non-polar solvent.
14. The method of item 13, wherein the non-polar solvent comprises an alkane.
15. The method of item 14, wherein the alkane is hexane, cyclohexane or heptane.
16. The method of item 15, wherein the alkane is cyclohexane.
17. The method of any one of the preceding items, wherein step (3) is performed without employing a solvent or diluent other than said polyethylene glycol (PEG).
18. The method of any one of the preceding items, wherein no halogenated solvent is used.
19. The method of any one of the preceding items, which does not include the use of any preparative chromatographic methods after step (3).
20. The method of any one of the preceding items, which does not include the use of any preparative chromatographic methods.
21. The method of any one of the preceding items, wherein the reacting in step (3) takes place for at least 10 minutes and for not more than 1 hour.
22. The method of any one of the preceding items, wherein the reacting in step (3) takes place for at least 20 minutes and for not more than 45 minutes.
23. The method of any one of the preceding items, wherein the reacting in step (3) takes place for 30 minutes.
24. The method of any one of the preceding items, wherein the reacting in step (3) takes place at a temperature of between 55° C. and 65° C.
25. The method of any one of the preceding items, wherein the reacting in step (3) takes place at a temperature of 60° C.
26. The method of any one of the preceding items, wherein in step (3), each molar equivalent (eq.) of said compound of said general Formula (XII) is reacted with at least 1.0, at least 2.0, at least 3.0 or at least 4.0 molar equivalents (eq.) of said polyethylene glycol (PEG).
27. The method of any one of the preceding items, wherein in step (3), each molar equivalent (eq.) of said compound of said general Formula (XII) is reacted with at least 4.5 molar equivalents (eq.) of said polyethylene glycol (PEG).
28. The method of any one of the preceding items, wherein in step (3), each molar equivalent (eq.) of said compound of said general Formula (XII) is reacted with at least 5.0 molar equivalents (eq.) of said polyethylene glycol (PEG).
29. The method of any one of the preceding items, wherein said polyethylene glycol (PEG) used in step (3) is characterized in that it is liquid at the reaction temperature of said step (3).
30. The method of any one of the preceding items, wherein the method is carried out at a scale which yields at least 100 g, at least 1 kg, at least 10 kg, at least 100 kg or at least 1000 kg of said compound of said Formula (XIX).
31. The method of any one of items 2-30, wherein the compound of formula (XII) obtained in step (2) is precipitated from a solvent.
32. The method of item 31, wherein the solvent is an alcohol solvent.
33. The method of any one of items 31-32, wherein the solvent is a polar solvent.
34. The method of any one of items 32-33, wherein the alcohol solvent is selected from the group consisting of isopropyl alcohol, ethanol, methanol and butanol or combinations thereof.
35. The method of any one of items 32-34, wherein the alcohol solvent is a secondary alcohol.
36. The method of any one of items 32-35, wherein the alcohol solvent is isopropyl alcohol.
37. The method of any one of items 32-33, wherein the solvent is acetonitrile.
38. The method of any one of items 2-37, wherein at least one recrystallization of said compound of Formula (XII) is carried out in between step (2) and step (3).
39. The method of item 38, wherein the solvent of any one of items 31 to 37 is employed as solvent for the recrystallization between step 2 and step 3.
40. The method of any one of the preceding items, wherein at least 1, at least 2, at least 3 or at least 4 aqueous washes are carried out after step 3.
41. The method of any one of the preceding items, wherein 4 to 6 aqueous washes are carried out after step 3.
42. A method of purifying a compound of formula (XIX),

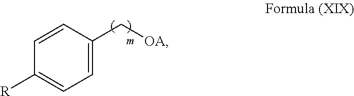

wherein m=1, R represents a hydrocarbon group having a linear chain of 2 to 12 carbon atoms and one or more methyl groups as substituents on said linear chain, and A represents a polyoxyethylene residue,
from a composition comprising the compound of formula (XIX), the method comprising subjecting a solution of said composition in a solvent selected from alkanes, ethers and esters and mixtures thereof to one or more aqueous washes.
43. The method of item 42, wherein said solvent selected from alkanes, ethers and esters and mixtures thereof is an ester.
44. The method of item 43, wherein said solvent selected from alkanes, ethers and esters and mixtures thereof is ethyl acetate.
45. The method of any of items 42 to 44, wherein the composition comprises polyethylene glycol (PEG).
46. The method of item 45, wherein the purifying of the compound of formula (XIX) comprises removing the polyethylene glycol (PEG) from the composition.
47. The method of item 46, wherein the removing of the polyethylene glycol (PEG) comprises said subjecting of the solution to the one or more aqueous washes.
48. The method of any one of items 42 to 47, wherein the composition further comprises a compound of the following general Formula (XIII):

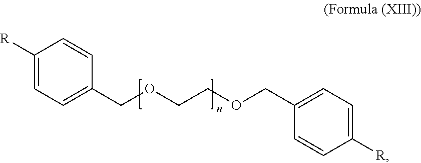

and wherein R is as defined in item 42 and n is an integer of n≥2.
49. The method of item 48, wherein the purifying of the compound of formula (XIX) comprises removing the compound of formula (XIII) from the composition.
50. The method of item 49, wherein the removing of the compound of formula (XIII) comprises preparing an aqueous solution comprising said compound of said general Formula (XIX) and removing said compound of formula (XIII) by one or more washes of said aqueous solution with a non-polar solvent.
51. The method of item 50, wherein the non-polar solvent comprises an alkane.
52. The method of item 51, wherein the alkane is hexane, cyclohexane or heptane.
53. The method of item 52, wherein the alkane is cyclohexane.
54. The method of any one of items 1 to 53, wherein the degree of purity of the compound of formula (XIX) as obtained by the method, as measured by high-performance liquid chromatography with UV detection at 200 nm, is at least 80%, preferably at least 85%, more preferably at least 90%, and most preferably at least 95%.
55. The method of any one of items 1 to 54, wherein the degree of purity of the compound of formula (XIX) as obtained by the method, as measured by high-performance liquid chromatography with detection using an evaporative light scattering detector (ELSD), is at least 99%.
56. The method of any one of the preceding items, further comprising a step of identifying a compound of the following general Formula (XIII):

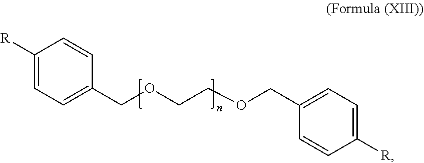

wherein R represents a hydrocarbon group having a linear chain of 2 to 12 carbon atoms and one or more methyl groups as substituents on said linear chain, and n is an integer of n≥2.
57. The method of any one of the preceding items, further comprising a step of quantifying a compound of the following general Formula (XIII):

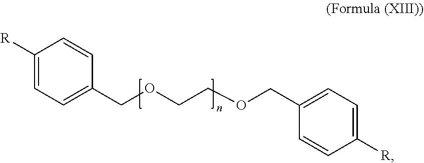

wherein R represents a hydrocarbon group having a linear chain of 2 to 12 carbon atoms and one or more methyl groups as substituents on said linear chain, and n is an integer of n≥2.
58. The method of any one of the preceding items, wherein A represents a polyoxyethylene residue comprising 4 to 16 oxyethylene units.
59. The method of any one of the preceding items, wherein A represents a polyoxyethylene residue comprising 8 to 10 oxyethylene units.
60. The method of any one of the preceding items, wherein A represents a polyoxyethylene residue comprising 9 or 10 oxyethylene units.
61. The method of any one of the preceding items, wherein R represents a hydrocarbon group having a linear chain of 2 to 6 carbon atoms and one or more methyl groups as substituents on said linear chain.
62. The method of any one of the preceding items, wherein R represents a hydrocarbon group having a linear chain of 2 to 6 carbon atoms and 2 to 4 methyl groups as substituents on said linear chain.
63. The method of any one of the preceding items, wherein R represents a hydrocarbon group having a linear chain of 4 carbon atoms and 4 methyl groups as substituents on said linear chain.
64. The method of any one of the preceding items, wherein R represents a 2,4,4-trimethyl-pent-2-yl group.
65. The method of any one of the preceding items, wherein the compound of Formula (XIX) is the following compound:

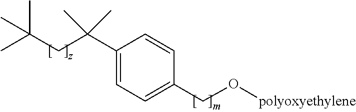

wherein m equals 1 and z is an integer selected from:
z=1 to 5.
66. The method of any one of the preceding items, wherein the compound of Formula (XIX) is the following compound:

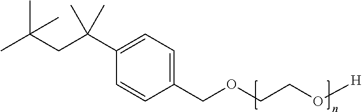

wherein n is an integer between 4 and 16, preferably wherein n is equal to 8, 9 or 10.
67. A compound of the following general Formula (XIX)

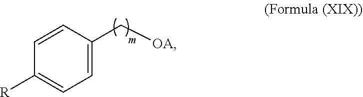

wherein m=1, R represents a hydrocarbon group having a linear chain of 2 to 12 carbon atoms and one or more methyl groups as substituents on said linear chain, and A represents a polyoxyethylene residue,
and wherein said compound is obtainable by the method of any one of the preceding items.
68. The compound of item 67, which exhibits a degree of purity, as measured by high-performance liquid chromatography with UV detection at 200 nm, of at least 80%, preferably at least 85%, more preferably at least 90%, and most preferably at least 95%.
69. The compound of item 67 or 68, which exhibits a degree of purity, as measured by high-performance liquid chromatography with detection using an evaporative light scattering detector (ELSD), of at least 99%.
70. Use of a non-polar solvent for extracting and removing a compound of the following general Formula (XIII):

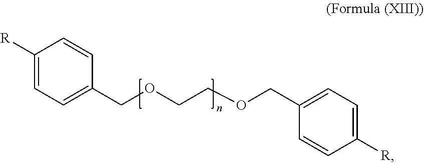

wherein R represents a hydrocarbon group having a linear chain of 2 to 12 carbon atoms and one or more methyl groups as substituents on said linear chain and n is an integer of n≥2,
from a composition comprising said compound of Formula (XIII) and a compound of formula (XIX),

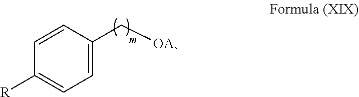

wherein m=1, R represents a hydrocarbon group having a linear chain of 2 to 12 carbon atoms and one or more methyl groups as substituents on said linear chain, and A represents a polyoxyethylene residue.
71. The use of claim 70, wherein the non-polar solvent is as defined in any one of items 51-53.
72. The use of item 70 or 71, wherein A is as defined in any one of items 58-60.
73. The use of any one of items 70 to 72, wherein R is as defined in any one of items 61-64.
74. The use of any one of items 70 to 73, wherein the compound of Formula (XIX) is as defined in any one of items 65-66.

### BRIEF DESCRIPTION OF THE DRAWINGS

FIG. 1: Virus inactivation efficiency of low concentrations of 4-tert-octylbenzyl alcohol polyethoxylate in S/D treatment of IVIG-containing liquid at 17° C.±1° C. A three-component mixture was used to give final concentrations of 0.04%-0.06% 4-tert-octylbenzyl alcohol polyethoxylate, 0.01%-0.02% Polysorbate 80, 0.01%-0.02% TnBP (side-by-side comparison with same concentration of Triton X-100). Virus inactivation over time is indicated by the virus reduction factor (RF) for two runs with PRV (A and B, respectively). Virus inactivation of S/D treatment using 4-tert-octylbenzyl alcohol polyethoxylate was compared to virus inactivation of S/D treatment using Triton X-100 (4-tert-octylphenol polyethoxylate, “TX-100”). Note that in (A), both detergents show identical inactivation kinetics. Filled symbols show values with remaining infectivity, non-filled symbols show reduction factors below the limit of detection.

FIG. 2: Virus inactivation efficiency of low concentrations of 4-tert-octylbenzyl alcohol polyethoxylate in S/D treatment of human serum albumin (HSA)-containing liquid at 1° C.±1° C. A three-component mixture was used to give final concentrations of 0.08%-0.1% 4-tert-octylbenzyl alcohol polyethoxylate, 0.02%-0.03% Polysorbate 80, 0.02%-0.03% TnBP (side-by-side comparison with same concentration of Triton X-100). Virus inactivation over time is indicated by the virus reduction factor (RF) for two runs with X-MuLV (A and B, respectively). Virus inactivation of S/D treatment using 4-tert-octylbenzyl alcohol polyethoxylate was compared to virus inactivation of S/D treatment using Triton X-100 (“TX-100”). Filled symbols show values with remaining infectivity, non-filled symbols show reduction factors below the limit of detection.

FIG. 3: Virus inactivation efficiency of low concentrations of 4-tert-octylbenzyl alcohol polyethoxylate in S/D treatment of HSA-containing liquid at 19° C.±1° C. A three-component mixture was used to give final concentrations of 0.08%-0.1% 4-tert-octylbenzyl alcohol polyethoxylate, 0.02%-0.03% Polysorbate 80, 0.02%-0.03% TnBP (side-by-side comparison with same concentration of Triton X-100). Virus inactivation over time is indicated by the virus reduction factor (RF) for two runs with X-MuLV (A and B, respectively). Virus inactivation of S/D treatment using 4-tert-octylbenzyl alcohol polyethoxylate was compared to virus inactivation of S/D treatment using Triton X-100 (“TX-100”). Filled symbols show values with remaining infectivity, non-filled symbols show reduction factors below the limit of detection.

FIG. 4: (A) Virus inactivation efficiency of low concentrations of 4-tert-octylbenzyl alcohol polyethoxylate or Triton X-100 reduced (i.e., 4-tert-octylcyclohexyl alcohol polyethoxylate) or Brij C10 (i.e. n-hexadecyl alcohol polyethoxylate) in detergent treatment of buffer containing HSA at 14° C.±1° C. A single-detergent treatment was used to give final concentrations of 0.09%-0.11% 4-tert-octylbenzyl alcohol polyethoxylate or Triton X-100 reduced or Brij C10 (side-by-side comparison with same concentration of Triton X-100). Virus inactivation over time is indicated by the mean virus reduction factor (RF) for two runs with BVDV. Virus inactivation of single-detergent treatment using 4-tert-octylbenzyl alcohol polyethoxylate or Triton X-100 reduced (“TX-100 red.”) or Brij C10 was compared to virus inactivation of single-detergent treatment using Triton X-100 (“TX-100”). Note that 4-tert-octylbenzyl alcohol polyethoxylate and Triton X-100 reduced have the same inactivation kinetics as Triton X-100. Filled symbols show values with remaining infectivity, non-filled symbols show reduction factors below the limit of detection. (B) Virus inactivation efficiency of low concentrations of 4-tert-octylbenzyl alcohol polyethoxylate or Triton X-100 reduced in detergent treatment of buffer containing HSA at 14C±1° C. A single-detergent treatment was used to give final concentrations of 0.02%-0.04% 4-tert-octylbenzyl alcohol polyethoxylate or Triton X-100 reduced (side-by-side comparison with same concentration of Triton X-100). Virus inactivation over time is indicated by the mean virus reduction factor (RF) for two runs with BVDV. Virus inactivation of single-detergent treatment using 4-tert-octylbenzyl alcohol polyethoxylate or Triton X-100 reduced (“TX-100 red.”) was compared to virus inactivation of single-detergent treatment using Triton X-100 (“TX-100”). Filled symbols show values with remaining infectivity, non-filled symbols show reduction factors below the limit of detection.

FIG. 5: (A) Virus inactivation efficiency of low concentrations of 4-tert-octylbenzyl alcohol polyethoxylate or Triton X-100 reduced in detergent treatment of IVIG-containing liquid at 17° C.±1° C. A single-detergent treatment was used to give final concentrations of 0.09%-0.11% 4-tert-octylbenzyl alcohol polyethoxylate or Triton X-100 reduced (side-by-side comparison with same concentration of Triton X-100). Virus inactivation over time is indicated by the mean virus reduction factor (RF) for two runs with BVDV. Virus inactivation of single-detergent treatment using 4-tert-octylbenzyl alcohol polyethoxylate or Triton X-100 reduced (“TX-100 red.”) was compared to virus inactivation of single-detergent treatment using Triton X-100 (“TX-100”). Filled symbols show values with remaining infectivity, non-filled symbols show reduction factors below the limit of detection. (B) Virus inactivation efficiency of low concentrations of 4-tert-octylbenzyl alcohol polyethoxylate or Triton X-100 reduced in detergent treatment of IVIG-containing liquid at 17° C.±1° C. A single-detergent treatment was used to give final concentrations of 0.02%-0.04% 4-tert-octylbenzyl alcohol polyethoxylate or Triton X-100 reduced (side-by-side comparison with same concentration of Triton X-100). Virus inactivation over time is indicated by the mean virus reduction factor (RF) for two runs with BVDV. Virus inactivation of single-detergent treatment using 4-tert-octylbenzyl alcohol polyethoxylate or Triton X-100 reduced (“TX-100 red.”) was compared to virus inactivation of single-detergent treatment using Triton X-100 (“TX-100”). Filled symbols show values with remaining infectivity, non-filled symbols show reduction factors below the limit of detection.

FIG. 6: Virus inactivation efficiency of low concentrations of 4-tert-octylbenzyl alcohol polyethoxylate or Triton X-100 reduced in detergent treatment of Factor VIII-containing liquid at 23° C.±1° C. A single-detergent treatment was used to give final concentrations of 0.09%-0.11% 4-tert-octylbenzyl alcohol polyethoxylate or Triton X-100 reduced (side-by-side comparison with same concentration of Triton X-100). Virus inactivation over time is indicated by the mean virus reduction factor (RF) for two runs with BVDV. Virus inactivation of single-detergent treatment using 4-tert-octylbenzyl alcohol polyethoxylate or Triton X-100 reduced (“TX-100 red.”) was compared to virus inactivation of single-detergent treatment using Triton X-100 (“TX-100”). Filled symbols show values with remaining infectivity, non-filled symbols show reduction factors below the limit of detection.

FIG. 7: Spectra of the product using HPLC method 1 as described in Example 3.

FIG. 8: Spectra of the product using HPLC method 2 as described in Example 3.

FIG. 9: Spectra of the product using HPLC method 3 as described in Example 3.

### DETAILED DESCRIPTION OF THE INVENTION

Unless otherwise defined below, the terms used in the present invention shall be understood in accordance with their common meaning known to the person skilled in the art.

All publications, patents and patent applications cited herein are hereby incorporated by reference in their entirety for all purposes.

### Definitions

The terms “virus having a lipid envelope”, “lipid-enveloped virus” and “enveloped virus” are used interchangeably herein, and have the meaning known to a person skilled in the art. For example, lipid-enveloped viruses can be Herpesviridae such as pseudorabies virus (PRV), herpes simplex virus, varicella-zoster virus, cytomegalovirus or Epstein-Barr virus; Hepadnaviridae such as hepatitis B virus; Togaviridae such as sindbis virus, rubella virus or alphavirus; Arenaviridae such as Imphocytic choriomeningitis virus; Flaviviridae such as West Nile virus, bovine viral diarrhea virus (BVDV), dengue virus, hepatitis C virus or yellow fever virus; Orthomyxoviridae such as influenza virus A, influenza virus B, influenza virus C, isavirus or thogotovirus; Paramyxoviridae such as sendai virus, measles virus, mumps virus, respiratory syncytial virus, Rinderpest virus or canine distemper virus; Bunyaviridae such as California encephalitis virus or hantavirus; Rhabdoviridae such as vesicular stomatitis virus or rabies virus; Filoviridae such as Ebola virus or Marburg virus; Coronaviridae such as corona virus or severe acute respiratory syndrome (SARS) coronavirus or SARS-CoV-2; Bornaviridae such as Borna disease virus; or Arteriviridae such as arterivirus or equine arteritis virus; Retroviridae such as Human Immunodeficiency Virus (HIV), Human T-lymphotropic virus 1 (HTLV-1) or xenotropic murine leukemia virus (X-MuLV); Poxviridae such as vaccinia virus or Orthopoxvirus variolae (Variolavirus).

The term “inactivating a virus having a lipid envelope” as used herein refers to disrupting the ability of the lipid-enveloped virus to infect cells. As will be understood by a person skilled in the art, the ability of a lipid-enveloped virus to infect cells, i.e. the infectivity of a lipid-enveloped virus, is typically assessed by determining the number of infectious virus particles in a liquid. Hence, the term “inactivating a virus having a lipid envelope” or “inactivating a lipid-enveloped virus” as used herein refers to reducing the number of infectious virus particles in a solution.

Herein the term “Log 10 reduction value” or “LRV” is used interchangeably with the term “virus reduction factor”, “reduction factor”, “RF” or “R”. In one embodiment, the “Log 10 reduction value” or “LRV” can be used as a measure of the reduction of infectious virus particles in a liquid. As used herein, the “Log 10 reduction value” or “LRV” is defined as the logarithm (base 10) of the ratio of infectious virus particles before virus inactivation to infectious virus particles after virus inactivation. The LRV value is specific to a given type of virus. It is evident for a skilled person in the art that any Log 10 reduction value (LRV) above zero is beneficial for improving the safety of methods and processes such as biopharmaceutical production methods and processes. The Log 10 reduction value (LRV) that is achieved by the methods according to the present invention is determined as known to a person skilled in the art. For example, the LRV can be determined by determining the number of infectious virus particles in a liquid before and after subjecting the liquid to the method for virus inactivation according to the present invention.

The skilled person will be aware of numerous methods to measure infectious virus particles in a liquid. For example, and without limitation, infectious virus particle concentrations in a liquid can preferably be measured by plaque assay or by the TCID₅₀ assay, more preferably by the TCID₅₀ assay. A “TCID₅₀ assay” as used herein refers to a tissue culture infectious dose assay. The TCID₅₀ assay is an end-point dilution test, wherein the TCID₅₀ value represents the viral concentration necessary to induce cell death or pathological changes in 50% of cell cultures inoculated.

The term “surfactant” as used herein refers to compounds that lower the surface tension between two liquids or between a liquid and a solid. Surfactants may act as detergents, wetting agents, emulsifiers, foaming agents, and dispersants.

In connection with the invention, terms such as “adding to”, “add to” or “added to” in connection with a first- and second-mentioned solvent, detergent and/or liquid encompass a situation where the first-mentioned solvent, detergent and/or liquid is added to the second-mentioned solvent, detergent and/or liquid. However, these terms are also meant to encompass a situation where the second-mentioned solvent, detergent and/or liquid is added to the first-mentioned solvent, detergent and/or liquid. Thus, terms such as “adding to”, “add to” or “added to” are not meant to specify whether the first-mentioned solvent, detergent and/or liquid is to be added to the second-mentioned solvent, detergent and/or liquid, or vice versa.

As will be known to a person skilled in the art, the term “detergent” is used according to its general meaning known in the art and includes in particular surfactants that can permeabilize lipid membranes. For example, the detergents Triton X-100 and deoxycholate have been suggested to solubilize amphipathic membrane proteins by binding to the hydrophobic segments of proteins (see Simons et al., 1973). Detergents are classified into three broad groups, depending on their electrical charge. Anionic detergents comprise an anionic, i.e. a negatively charged, hydrophilic group. Exemplary anionic detergents are tetradecyltrimethylammonium bromide, dodecyltrimethylammonium bromide, sodium laureth sulphate, sodium dodecyl sulphate (SDS), cetrimide and hexadecyltrimethylammonium bromide. Cationic detergents comprise a cationic, i.e. a positively charged, hydrophilic group. Exemplary cationic detergents are benzalkonium chloride, cetyl trimethlammonium bromide (CTAB), cetylpyridinium chloride (CPC) and benzethonium chloride (BZT). The term “non-ionic detergents” as used herein refers to detergents having no positive or negative charge. Exemplary non-ionic detergents are sorbitan esters (sorbitan monolaurate, sorbitan monopalmitate, sorbitan monostearate, sorbitan tristearate, sorbitan monooleate, Sorbitan trioleate), polysorbates (polyoxyethylene (20) sorbitan monolaurate (Polysorbate 20), polyoxyethylene (20) sorbitan monopalmitate, polyoxyethylene (20) Sorbitan monostearate, polyoxyethylene (20) sorbitan tristearate, polyoxyethylene (20) Sorbitan trioleate, Polyoxyethylen(20)-sorbitan-monooleate (Tween 80/Polysorbate 80)), poloxamers (poloxamer 407, poloxamer 188) and cremophor. Detergents in accordance with the invention are as specified in the preferred embodiments.

The term “non-phenolic” as used herein is interchangeably used with the term “phenol-free”. A non-phenolic detergent according to the present invention refers to a detergent that does not contain any phenol functional groups.

The term “aromatic” has the meaning known to a person skilled in the art. A detergent that is not aromatic refers to a detergent that does not contain any aromatic rings.

The term “environmentally compatible” as used herein has the meaning known to a person skilled in the art. In a preferred embodiment of the invention, with regard to detergents, the term “environmentally compatible” indicates that the detergent does not behave as an endocrine disrupter. Endocrine disruptors are exogenous substances that alter function(s) of the endocrine system and consequently cause adverse health effects in an intact organism or its progeny, or (sub)populations. The skilled person will be aware of various methods to identify endocrine disruptors. Further information on endocrine disruptors and their evaluation can be found, e.g., in the ECHA's “Support document for identification of 4-(1,1,3,3-tetramethylbutyl)phenol, ethoxylated as substances of very high concern because, due to their degradation to a substance of very high concern (4-(1,1,3,3-tetramethylbutyl)phenol) with endocrine disrupting properties, they cause probable serious effects to the environment which give rise to an equivalent level of concern to those of CMRs and PBTs/vPvBs”, adopted on 12 Dec. 2012, which is hereby incorporated in its entirety and for all purposes; or in the brochure “Global Assessment of the State-of-the-Science of Endocrine Disruptors” (WHO/PCS/EDC/02.2), which is hereby incorporated in its entirety and for all purposes, published by the International Programme on Chemical Safety of the World Health Organization.

The term “biological medicinal product” as used herein is known in the art and refers to a product, the active substance of which is a biological substance, e.g. a biological substance which is produced by mammalian cells or microorganisms. As used herein, the biological medicinal product used in the methods of the invention is not limited to the final manufactured product but preferably also includes intermediate products at any stage of the manufacturing process.

The term “biopharmaceutical drug” as used herein has the meaning known to a person skilled in the art.

Biopharmaceutical drugs include both recombinant biopharmaceutical drugs and biopharmaceutical drugs obtained from other sources such as human plasma.

As used herein the term “depth filter” has the meaning known in the art. In particular, such a filter (e.g., gradient-density depth filter) achieves filtration within the depth of the filter material. A common class of such filters is those that comprise a random matrix of fibers bonded (or otherwise fixed), to form a complex, tortuous maze of flow channels. Particle separation in these filters generally results from entrapment by, or adsorption to, the fiber matrix. Depth filters can be composed of numerous materials including, without limitation, a fibrous bed of cellulose or polypropylene fibers, fiber mats, woven or nonwoven fabrics, or a synthetic fabric, such as nylon or rayon, e.g., Miracloth® (Calbiochem, La Jolla, Calif.) along with a filter aid or “matrix”, e.g., paper, plastic, metal, glass, glass fibers, nylon, polyolefin, carbon, ceramics, diatomaceous earth, cellulose or diatomite (skeletal remains of minute algae (diatoms) found in marine deposits that have lifted above sea level).

The term “purifying a biopharmaceutical drug” as used herein has the meaning known to a person skilled in the art, and refers to separating the biopharmaceutical drug from other substances that may be comprised in the mixture of the present invention. In a preferred embodiment of the invention, the term “purifying a biopharmaceutical drug” refers to separating the biopharmaceutical drug from the detergent of the present invention.

The term “chromatography” is used according to its meaning known in the art. It includes any chromatography technique which separates an analyte of interest (e.g. a target molecule such as a biopharmaceutical drug) from other molecules present in a mixture. Usually, the analyte of interest is separated from other molecules as a result of differences in rates at which the individual molecules of the mixture migrate through a stationary medium under the influence of a moving phase, or in bind and elute processes.

The terms “chromatography resin” and “chromatography media” are used interchangeably herein and refer to any kind of phase (e.g., a solid phase) which separates an analyte of interest (e.g., a target molecule such as a biopharmaceutical drug) from other molecules present in a mixture. Usually, the analyte of interest is separated from other molecules as a result of differences in rates at which the individual molecules of the mixture migrate through a stationary solid phase under the influence of a moving phase, or in bind and elute processes. Examples of various types of chromatography media include, for example, cation exchange resins, cation exchange membranes, affinity resins, anion exchange resins, anion exchange membranes, hydrophobic interaction resins and ion exchange monoliths.

The term “pharmaceutical formulation” as used herein has the meaning known to a person skilled in the art and refers to any formulation that is suitable for administration to a patient. Pharmaceutical formulations can be prepared according to methods known in the art. For example, for any biopharmaceutical drug that is present in the formulation, a skilled person will be able to choose and add preferred additional ingredients including buffers, stabilizers, surfactants, anti-oxidants, chelating agents and/or preservatives etc.

The term “solvent/detergent mixture” as used herein has the meaning known to the person skilled in the art. In a preferred embodiment, the solvent/detergent mixture used in accordance with the invention contains at least one solvent other than water and at least one detergent. The solvent used in accordance with the invention is preferably an organic solvent, and is most preferably tri-n-butyl phosphate. The number of different solvents and/or detergents contained in the mixture is not particularly limited. For example, the solvent/detergent mixture can be composed of tri-n-butyl phosphate, Polysorbate 80 and a polyoxyethylene ether detergent according to the present invention.

It is to be understood that the term “between” when used to indicate a numerical range in the present invention includes the indicated lower and upper limits of the respective range. For example, when a temperature is indicated to be between 0° C. and 10° C., this includes the temperatures of 0° C. and 10° C. Similarly, when a variable x is indicated to be an integer between, e.g., 4 and 16, this includes the integers 4 and 16.

As used herein, each occurrence of terms such as “comprising” or “comprises” may optionally be substituted with “consisting of” or “consists of”.

### Embodiments

The present invention pertains to polyoxyethylene and polyoxypropylene ether detergents and to methods for their preparation and purification, and to methods and uses of these detergents for virus inactivation.

The toxic activity of Triton-X100 arises from its phenol moiety, which has the ability to dock in certain endocrine receptors of marine life organisms. By inserting a methylene group between the aromatic ring and the PEG chain, the phenol functionality of Triton-X100 is no longer present in the new structure of Formula XIX. In addition, once the PEG chain breaks down after being released in in the environment the revealed benzyl alcohol will easily oxidize to the corresponding benzoic acid. This metabolite has a completely different polarity and geometric structure than a phenol derivative, which will reduce binding to endocrine receptors.

Based on the above considerations, the present inventors synthesized non-phenolic polyoxyethylene ethers and tested their antiviral activity (see Examples 1 to 9).

Thus, in one aspect, the present invention provides non-phenolic polyoxyethylene ethers of the following general Formula (XIX):

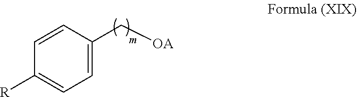

wherein m=1, R represents a hydrocarbon group having a linear chain of 2 to 12 carbon atoms and one or more methyl groups as substituents on said linear chain, and A represents a polyoxyethylene residue.

The present invention also provides non-phenolic polyoxyethylene ethers of the following general Formula (XIX):

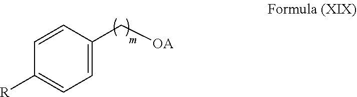

wherein m=1, R represents a hydrocarbon group having a linear chain of 2 to 12 carbon atoms and one or more methyl groups as substituents on said linear chain, and A represents a polyoxyethylene residue, optionally with the proviso that 29-[4-(1,1,3,3-tetramethylbutyl)phenyl]-3,6,9,12,15,18,21,24,27-nonaoxanonacosan-1-ol having the following structural formula is excluded.

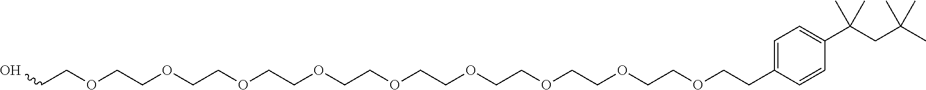

In Formula (XIX) R represents a hydrocarbon group having a linear chain of 2 to 12, preferably 2 to 8, more preferably 2 to 6 and most preferably 4 carbon atoms and one or more, preferably 2 to 6, and most preferably 4 methyl group(s) as substituent(s) on said linear chain.

Preferably R represents a hydrocarbon group having a linear chain of 2 to 12, preferably 2 to 8, more preferably 2 to 6 and most preferably 4 carbon atoms and 2 methyl group(s) as substituent(s) on said linear chain; a hydrocarbon group having a linear chain of 2 to 12, preferably 2 to 8, more preferably 2 to 6 and most preferably 4 carbon atoms and 4 methyl group(s) as substituent(s) on said linear chain; or a hydrocarbon group having a linear chain of 2 to 12, preferably 2 to 8, more preferably 2 to 6 and most preferably 4 carbon atoms and 6 methyl group(s) as substituent(s) on said linear chain.

Most preferably R represents a 2,4,4-trimethyl-pent-2-yl group.

In Formula (XIX) A represents a polyoxyethylene residue, preferably a polyoxyethylene residue comprising 2-20 oxyethylene units, more preferably a polyoxyethylene residue comprising 4 to 16 oxyethylene units, even more preferably a polyoxyethylene residue comprising 8 to 12 oxyethylene units, and most preferably a polyoxyethylene residue comprising 9 or 10 oxyethylene units.

A preferred embodiment of the compound of general Formula (XIX) is a compound wherein R represents a hydrocarbon group having a linear chain of 2 to 6 carbon atoms and 2 to 4 methyl groups as substituents on said linear chain; m=1, and A represents a polyoxyethylene residue comprising 8 to 12 oxyethylene units.

A specific preferred embodiment of the compound of general Formula (XIX) is the following compound:

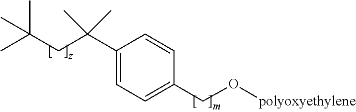

wherein m equals 1 and z is an integer selected from:
z=1 to 5.

Another specific preferred embodiment of the compound of general Formula (XIX) is the following compound:

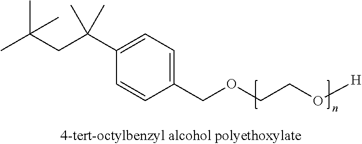

wherein n is an integer between 4 and 16, preferably wherein n is equal to 8, 9 or 10.

Polyoxyethylene ethers are known to a person skilled in the art and have the following structure according to Formula A:

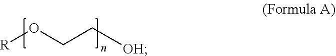

wherein n is equal to or higher than one.

As will be clear to a person skilled in the art, polyoxypropylene ethers can have very similar properties as the polyoxyethylene ethers in accordance with the present invention and can be prepared and purified in a corresponding manner. Thus, in accordance with all other embodiments of the invention, e.g. in all methods of the present invention, the polyoxyethylene ethers may be replaced by polyoxypropylene ethers.

Polyoxypropylene ethers are also known to a person skilled in the art and have the following structure according to Formula B:

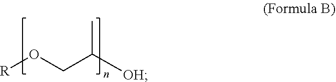

wherein n is an integer of n 2.

Thus, in a further aspect, the present invention provides non-phenolic polyoxypropylene ethers of the following general Formula (XIX):

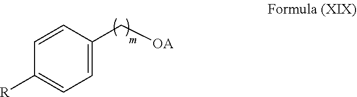

wherein m=1, R represents a hydrocarbon group having a linear chain of 2 to 12 carbon atoms and one or more methyl groups as substituents on said linear chain, and A represents a polyoxypropylene residue.

In Formula (XIX) R represents a hydrocarbon group having a linear chain of 2 to 12, preferably 2 to 8, more preferably 2 to 6 and most preferably 4 carbon atoms and one or more, preferably 2 to 6, and most preferably 4 methyl group(s) as substituent(s) on said linear chain.

Preferably R represents a hydrocarbon group having a linear chain of 2 to 12, preferably 2 to 8, more preferably 2 to 6 and most preferably 4 carbon atoms and 2 methyl group(s) as substituent(s) on said linear chain; a hydrocarbon group having a linear chain of 2 to 12, preferably 2 to 8, more preferably 2 to 6 and most preferably 4 carbon atoms and 4 methyl group(s) as substituent(s) on said linear chain; or a hydrocarbon group having a linear chain of 2 to 12, preferably 2 to 8, more preferably 2 to 6 and most preferably 4 carbon atoms and 6 methyl group(s) as substituent(s) on said linear chain.

Most preferably R represents a 2,4,4-trimethyl-pent-2-yl group.

In Formula (XIX) of this further aspect of the invention, A represents a polyoxypropylene residue, preferably a polyoxypropylene residue comprising 2-20 oxypropylene units, more preferably a polyoxypropylene residue comprising 4 to 16 oxypropylene units, even more preferably a polyoxypropylene residue comprising 8 to 12 oxypropylene units, and most preferably a polyoxypropylene residue comprising 9 or 10 oxypropylene units.

A preferred embodiment of the compound of general Formula (XIX) is a compound wherein R represents a hydrocarbon group having a linear chain of 2 to 6 carbon atoms and 2 to 4 methyl groups as substituents on said linear chain; m=1, and A represents a polyoxypropylene residue comprising 8 to 12 oxypropylene units.

As referred to herein, a “polyoxyethylene ether” in accordance with the invention is preferably a polyoxyethylene ether according to its common meaning in the art. Alternatively, a polyoxyethylene ether in accordance with the invention may also be a polyoxyether, where a part of, preferably the majority of, the total number of polyoxyether molecules are polyoxyethylene ether molecules, but where another part of, preferably the minority of, the total number of polyoxyether molecules are mixed polymers comprising oxyethylene and oxypropylene units and/or polymers comprising oxypropylene units. In this case, the term “the majority of the total number of polyoxyether molecules are polyoxyethylene ether molecules” means that at least 50% of the total number of polyoxyether molecules are polyoxyethylene ether molecules. Preferably, at least 60% of the total number of polyoxyether molecules are polyoxyethylene ether molecules. More preferably, at least 70% of the total number of polyoxyether molecules are polyoxyethylene ether molecules. Still more preferably, at least 80% of the total number of polyoxyether molecules are polyoxyethylene ether molecules. Still more preferably, at least 90% of the total number of polyoxyether molecules are polyoxyethylene ether molecules. Most preferably, at least 95% of the total number of polyoxyether molecules are polyoxyethylene ether molecules. Yet alternatively, a polyoxyethylene ether in accordance with the invention may also be a mixed polyoxyether, comprising a majority (e.g. at least 60%, preferably at least 70%, more preferably at least 80%, still more preferably at least 90%, and most preferably at least 95%) of oxyethylene units and a minority of oxypropylene units.

The detergents in accordance with the present invention are non-phenolic.

According to the invention, the compound of Formula (XIX) may be prepared and purified as indicated in the preferred embodiments.

Thus, the invention encompasses a method for preparing and purifying a compound of the following general Formula (XIX)

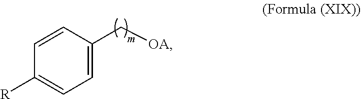

wherein m=1, R represents a hydrocarbon group having a linear chain of 2 to 12 carbon atoms and one or more methyl groups as substituents on said linear chain, and A represents a polyoxyethylene residue,
the method comprising the steps of
(3) Reacting a compound of the following general Formula (XII)

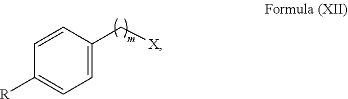

wherein m and R are as defined above and X represents a halogen atom or a hydroxyl group,
with a polyethylene glycol (PEG) of the general formula HO(CH₂CH₂O)ₙH, wherein n is an integer of n≥2, to produce a compound of said general formula (XIX) and a side product of the following general Formula (XIII)

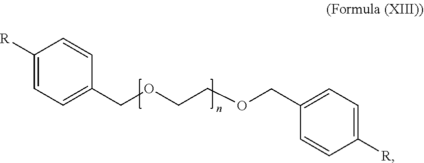

wherein R is as defined above and n is an integer of n≥2;
and
(4) Purifying the compound of said general Formula (XIX) by removing therefrom unreacted polyethylene glycol (PEG) and said side product.

The method may further comprise the following step (2) prior to said step (3):
(2) Converting a compound of the following general Formula (XIV),

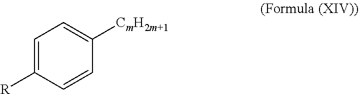

wherein m and R are as defined in claim 1,
into a compound of the following general Formula (XII)

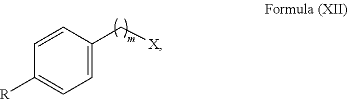

wherein m, R and X are as defined in claim 1.

Additionally, the method may further comprise the following step (1) prior to said step (2):
(1) Reacting toluene so as to obtain a compound of the following general Formula (XIV)

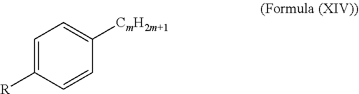

wherein m and R are as defined in claim 1.

This preferred method of the invention is a modification of the Scheme 2 shown below. Said method of the invention offers the advantage of requiring only three steps for the synthesis, not requiring chromatographic purification, thereby reducing the volume of required solvents, avoiding the use of expensive (e.g. Tf₂O, Pd catalyst), toxic (e.g. octylphenol, Zn(CN)₂, DMF, MsCl) and hazardous (e.g. LiAlH₄) chemicals, and being amenable to implementation on a large scale.

Acids that are commonly employed in Friedel-Craft alkylations, such as sulfuric acid, may be used as a catalyst of the reaction in step (1). The catalyst is preferably a perfluoroalkylsulfonic acid, more preferably heptafluoro-1-proanesulfonic acid, trifluoromethanesulfonic acid (triflic acid) or nonafluoro-1-butanesulfonic acid.

Halogenation reagents which can be used in the conversion in step (2) may be selected from halogenation reagents known in the art. Preferred reagents include
an N-halosuccinimide such as N-bromosuccinimide (NBS).
bromine,
suitable bromine complex reagents such as bromine-1,4-dioxane complex,
suitable tribromide complex reagents such as tetrabutylammonium tribromide, trimethylphenylammonium tribromide, benzyltrimethylammonium tribromide, pyridinium bromide perbromide, 4-dimethylaminopyridinium bromide perbromide, 1-butyl-3-methylimidazolium tribromide and 1,8-diazabicyclo[5.4.0]-7-undecene hydrogen tribromide,
dibromoisocyanuric acid,
tribromoisocyanuric acid,
1,2-dibromo-1,1,2,2-tetrachloroethane,
N-bromophthalimide, and
hypobromite salts.

The most preferred halogenation reagent is N-bromosuccinimide (NBS).

Radical initiators which can be used in the conversion in step (2) may be suitably selected from physical and chemical radical initiators known in the art. They include, for instance, light, AIBN (azobis(isobutyronitrile), benzoyl peroxide, di-tert-butyl peroxide, methyl ethyl ketone peroxide, acetone peroxide, peroxydisulfate salts or one of their derivatives. Most preferred is AIBN (azobis(isobutyronitrile).

Preferred solvents which can be used for the conversion in step (2) are cyclohexane and acetonitrile. Heptane, ethylacetate, CCl₄ and benzene can also be used as solvents for the conversion in step (2).

In a preferred embodiment of the invention, the compound of formula (XII) obtained in step (2) is precipitated from a solvent. Preferably, the solvent is a polar solvent. In a preferred embodiment, the solvent is an alcohol solvent which is preferably selected from the group consisting of isopropyl alcohol, ethanol, methanol and butanol or combinations thereof. More preferably, the alcohol solvent is a secondary alcohol such as isopropyl alcohol. In another preferred embodiment, the solvent is acetonitrile. Water increases the polarity of solvents. Therefore, a polar solvent used in accordance with invention may also comprise water. Thus, a polar solvent used in accordance with the invention may, for instance, be a solvent mixture comprising (i) water, and (ii) an alcohol as indicated above and/or acetonitrile.

Alcoholic solvents may react with a halogenated intermediate according to formula (XII) to form an ether side-product. Primary alcohols are prone to this reaction, whereas secondary alcohol such as isopropanol are less prone to form this side product and are therefore advantageous. Non-alcoholic solvents such as acetonitrile can also be used to avoid such an ether side product and are therefore also advantageous.

Polar solvents are known in the art. Solvents with a dielectric constant of higher than 15.0 are generally considered to be polar. Examples of such polar solvents are isopropyl alcohol, ethanol, methanol and butanol, as well as acetonitrile. As used herein, values of dielectric constants referred to herein refer to the dielectric constants measured at a reference temperature of 20° C.

It is understood that a polyethylene glycol (PEG) which can be used in step (3) can suitably be selected by a person skilled in the art. Such a polyethylene glycol (PEG) is not particularly limited. Preferably, the polyethylene glycol (PEG) is characterized in that it is liquid at the reaction temperature of said step (3).

Preferred polyethylene glycols (PEGs) which can be used in step (3) include PEGs of an average molecular weight of between 150 and 1000 Daltons. More preferably, a polyethylene glycol (PEG) which can be used in step (3) is selected from the group consisting of PEG200, PEG400, PEG600, PEG800 and PEG1000. Most preferably, the polyethylene glycol (PEG) is PEG400.

A compound of the general Formula (XIII) is obtained as a side product in the PEGylation reaction in step (3). In step (4), the compound of the general Formula (XIX) is purified by removing therefrom said side product of Formula (XIII). Based on the knowledge of the compounds of Formula (XIX) and Formula (XIII) provided by the present invention, and based on the differences between these two compounds, it is understood that the skilled person will be able to remove the side product of the general Formula (XIII) by appropriate method steps. For example, since the side product of the general Formula (XIII) is bifunctional, it is larger and less polar than the compound of the general Formula (XIX). Accordingly, in step (4), the compound of the general Formula (XIX) can be purified by removing therefrom said side product using a size-based and/or a polarity-based purification method. In a preferred embodiment, the purifying of said compound of said general Formula (XIX) by removing therefrom said side product in step (4) comprises removing said side product from the compound of said general Formula (XIX) by subjecting a solution of said compound of said general Formula (XIX) in a mixture comprising water and a water-miscible organic solvent to one or more washes with a non-polar solvent.

Water-miscible organic solvents as used herein are known in the art and comprise inter alia the following solvents: acetaldehyde, acetic acid, acetone, acetonitrile, 1,2-butanediol, 1,3-butanediol, 1,4-butanediol, 2-butoxyethanol, butyric acid, diethanolamine, diethylenetriamine, dimethylformamide, dimethoxyethane, dimethyl sulfoxide, 1,4-dioxane, ethanol, ethylamine, ethylene glycol, formic acid, furfuryl alcohol, glycerol, methanol, methyl diethanolamine, methyl isocyanide, N-Methyl-2-pyrrolidone, 1-propanol, 1,3-propanediol, 1,5-pentanediol, 2-propanol (isopropanol), propanoic acid, propylene glycol, pyridine, tetrahydrofuran and triethylene glycol.

Preferably, said solution of said compound of said general Formula (XIX) in a mixture comprising water and a water-miscible organic solvent is a solution of said compound of said general Formula (XIX) in a mixture of alcohol and water. More preferably, said solution of said compound of said general Formula (XIX) is a solution of said compound of said general Formula (XIX) in a mixture of ethanol and water. It is understood that suitable mixing ratios of such mixtures can readily be determined by a person skilled in the art. An exemplary mixing ratio of the mixture of ethanol and water is between 50:1 (v/v) and 10:1 (v/v), preferably 20:1 (v/v).

Non-polar solvents are known in the art. Solvents with a dielectric constant of less than 15.0 are generally considered to be nonpolar. Preferably, a non-polar solvent used for said washes has a dielectric constant of less than 5.0, more preferably less than 2.5. Examples of such more preferred solvents are hexane, cyclohexane and heptane. As used herein, values of dielectric constants referred to herein refer to the dielectric constants measured at a reference temperature of 20° C.

The invention also provides a method of purifying a compound of formula (XIX),

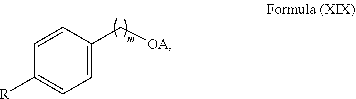

wherein m=1, R represents a hydrocarbon group having a linear chain of 2 to 12 carbon atoms and one or more methyl groups as substituents on said linear chain, and A represents a polyoxyethylene residue,
from a composition comprising the compound of formula (XIX), the method comprising subjecting a solution of said composition in a solvent selected from alkanes, ethers and esters and mixtures thereof to one or more aqueous washes.

Preferably, the composition further comprises a compound of the following general Formula (XIII):

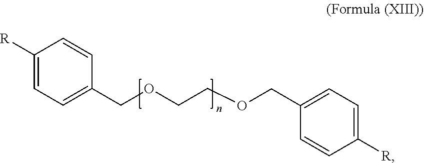

wherein R is as defined in claim 37 and n is an integer of n≥2. Preferably, the purifying of the compound of formula (XIX) comprises removing the compound of formula (XIII) from the composition. More preferably, the removing of the compound of formula (XIII) comprises preparing an aqueous solution comprising said compound of said general Formula (XIX) and removing said compound of formula (XIII) by one or more washes of said aqueous solution with a non-polar solvent. Non-polar solvents are as defined above, and aqueous solutions which can be used in accordance with the invention comprise water and are preferably solutions of said compound of said general Formula (XIX) in a mixture comprising water and a water-miscible organic solvent. Preferred solutions of said compound of said general Formula (XIX) in a mixture comprising water and a water-miscible organic solvent are as defined above.

In a preferred embodiment of the methods of the invention, these methods comprise a step of identifying and/or a step of quantifying a compound of the following general Formula (XIII):

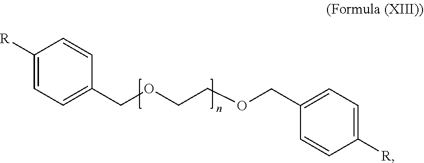

wherein R represents a hydrocarbon group having a linear chain of 2 to 12 carbon atoms and one or more methyl groups as substituents on said linear chain, and n is an integer of n≥2. Such identification may be performed by any suitable methods for the structural identification of chemical compounds known in the art, including, for example, ¹H-NMR measurements. Other NMR measurements such as ¹¹C-NMR or 2D NMR can also be used for the identification. In addition, HRMS (high resolution mass spectrometry) may also be used for the identification. A quantification of the compound of said Formula (XIII) can be performed by any suitable methods for the quantitation of chemical compounds known in the art, including, for example, high-performance liquid chromatography with UV detection and high-performance liquid chromatography with detection using an evaporative light scattering detector (ELSD).

Generally, in accordance with the invention, high-performance liquid chromatography with UV detection is preferably carried out using UV detection at 200 nm. More preferably, in accordance with the invention, high-performance liquid chromatography with UV detection is preferably carried out using the method settings indicated in the HPLC methods 1 and 2 of Example 3, more preferably using the method settings indicated in the HPLC method 2 of Example 3.

Preferably, in accordance with the invention, high-performance liquid chromatography with detection using an evaporative light scattering detector (ELSD) is carried out using the method settings indicated in the HPLC method 2 of Example 3.

The settings which are used for the HPLC method 2 of Example 3 are as follows:
HPLC: Agilent 1260
Column: Zorbax 300SB-C3, 5 μm, 2.1×150 mm (V=0.520 mL)
Eluents: water (A), acetonitrile (B)
Gradient elution: gradient elution: 35% B (0 min)→35% B (4 min)→97% B (20 min)→97% B (2 5 min)
Post time: 8 min
Flow: 0.5 mL/min
Column temperature: 25° C.
Injection volume: 5 μL
Detection: UV at a wavelength of 200 nm, or ELSD

The preferred methods of the invention for the preparation and purification of the detergents of the invention offer, inter alia, the following advantages:
No chromatographic purification, the overall usage of organic solvent is reduced drastically.
No hazardous solvent such as dichloromethane is required.
In addition, the purification procedure can be performed in short time; the overall synthesis cost is minimized.
No solvent needed in the 3rd step: The procedure is simple to perform, and the number of different solvents present during the work-up can be minimized.
The overall yield is comparable to that of other methods, but the process is more efficient than such methods in the sense that it is cheaper to carry out, can be performed faster and results in less waste.
The amount of bifunctional side product can be effectively reduced by several means, e.g. by extraction of the product with a non-polar solvent such as hexane or cyclohexane, and/or by using high (e.g. at least 4, at least 4.5 or at least 5) molar equivalents of PEG during step (3).
For the above reasons, the preferred methods of the invention can easily be carried out at industrial scale, thereby allowing for the production of kilogram amounts of pure product using standard equipment.

Alternatively, the polyoxyethylene ether detergents in accordance with the present invention can, for instance, be synthetized by ethoxylation reaction. Ethoxylation is an industrial process performed upon alcohols in order to generate alcohol ethoxylates (aka polyoxyethylene ethers). The reaction proceeds by passing ethylene oxide through the alcohol at high temperature (e.g., around 180° C.) and under high pressure (e.g., under 1-2 bar of pressure), with a base (such as potassium hydroxide, KOH) serving as a catalyst. The process is highly exothermic.

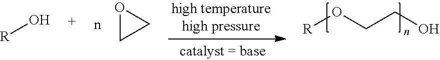

The reaction leads to the formation of a product with a wide polydispersity of repeat unit length (the value of n in the equation above is an average polymer length).

While the compounds of the invention can be polyoxyethylene ethers (=ethoxylation reaction is performed with ethylene oxide gas), it is also possible to perform a similar reaction on an industrial scale using propylene oxide (=propoxylation). The only difference would be methyl group substitution in the PEG chain:

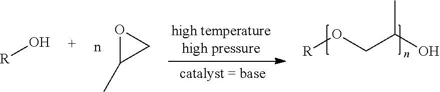

Ethoxylation and propoxylation can also be combined in the same process and lead to a mixed product, such as a mixed polymer polyoxyether comprising oxyethylene and oxypropylene units.

On a laboratory scale, polyoxyethylene ether detergents can be produced by first forming a good leaving group (such as Cl, Br, I, OMs or OTf) from the hydroxyl group of the alcohol and then reacting this substrate with a mono deprotonated polyethylene glycol. Alternatively, a good leaving group (such as Cl, Br, I, OMs or OTf) can be installed on the polyethylene glycol chain that is reacted in a second step with a deprotonated alcohol.

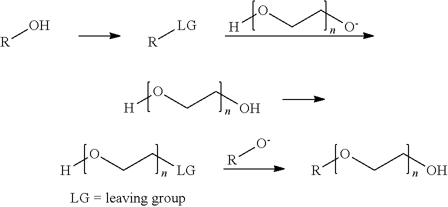

Exemplary methods for the synthesis of polyoxyethylene ethers are described in detail, e.g., in U.S. Pat. No. 1,970,578 and Di Serio et al. (2005), which are herewith incorporated by reference in their entirety for all purposes.

The compound of Formula (XIX) can also be synthesized by commonly known synthetic methods, such as those described in Vogel's Textbook of Practical Organic Chemistry (5th Edition, 1989, A. I. Vogel, A. R. Tatchell, B. S. Furnis, A. J. Hannaford, P. W. G. Smith). For example, 4-tert-octylbenzyl alcohol polyethoxylate can be prepared by converting a phenol comprising a substituent corresponding to group R of general formula (XIX) into the corresponding benzoic acid or homobenzoic acid, reducing the acid group to an alcohol group and reacting the alcohol to form a polyethylene glycol ether (also known as polyoxyethylene ether; POE ether). Herein, ethylene oxide or a suitable polyethylene glycol can serve as a basis for introducing the polyethylene glycol ether functionality (Scheme 1; representative examples of experimental procedures that are effective for carrying out individual transformations can be found, for example, in Bioorganic & Medicinal Chemistry, 16(9), 4883-4907, 2008; Journal of Medicinal Chemistry, 48(10), 3586-3604, 2005; Journal of Physical Chemistry B, 107(31), 7896-7902; 2003; Journal of Nanoparticle Research, 15(11), 2025/1-2025/12, 12 pp., 2013; and PCT Int. Appl., 2005016240, 24 Feb. 2005.).

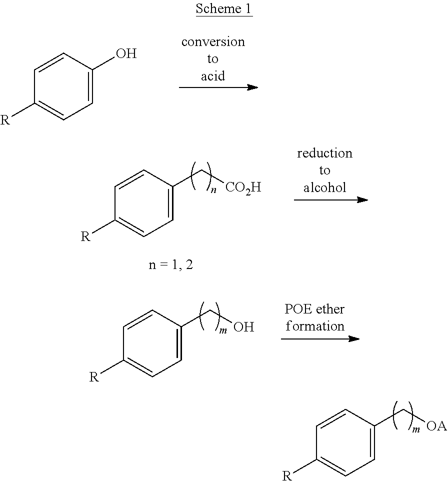

Alternatively, the compound of General Formula (XIX) can be accessed through the alkylation of toluene, followed by functionalization of the benzylic methyl group and further reaction to form a polyethylene glycol ether (Scheme 2; representative examples of experimental procedures that are effective for carrying out individual transformations can be found, for example, in Russian Journal of Applied Chemistry, 82(6), 1029-1032, 2009; Journal of Organic Chemistry, 79(1), 223-229, 2014; and Chemistry—A European Journal, 23(60), 15133-15142, 2017)). The direct alkylation of benzyl alcohol to obtain a suitable intermediate for introducing the polyethylene glycol ether functionality also is envisaged (Scheme 3; representative examples of experimental procedures for corresponding transformations can be found, for example, in Russian Journal of Organic Chemistry, 51(11), 1545-1550, 2015).

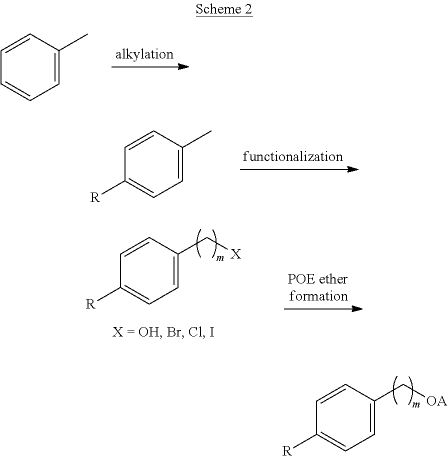

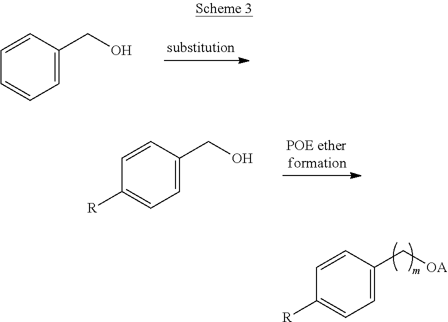

The above polyoxyethylene ethers with the structure according to Formula (XIX) can be used in the methods for inactivating a virus having a lipid envelope according to the present invention.

As described above, and as will be clear to a person skilled in the art, the synthesis of polyoxyethylene ethers usually yields products with a wide polydispersity of polyoxyethylene repeat unit lengths. Hence, when the number of polyoxyethylene repeat units is indicated for the polyoxyethylene ethers of the present invention, this number refers to the average polyoxyethylene repeat unit length, rounded to the nearest integer. The average polyoxyethylene repeat unit length refers to the average number of polyoxyethylene repeat units of all polyoxyethylene ether molecules of a sample, rounded to the nearest integer. The same applies to polyoxypropylene ethers. For example, if, in formulae used in accordance with the invention, the number of polyoxyethylene or polyoxypropylene repeat units is n=10, this means that the average number of polyoxyethylene or polyoxypropylene repeat units of all polyoxyethylene or polyoxypropylene ether molecules, rounded to the nearest integer, is 10.

The polyoxyethylene or polyoxypropylene ether detergents used in the methods for inactivating a virus having a lipid envelope according to the present invention have an average number of polyoxyethylene or polyoxypropylene repeat units of 2 to 100, 2 to 50, 2 to 20, or 4 to 16, or 9 or 10. Preferably, the average number of polyoxyethylene or polyoxypropylene repeat units is 4 to 16, more preferably 9 or 10, and most preferably 10.

As will be clear to a person skilled in the art, in the polyoxyethylene/polyoxypropylene ether detergents in accordance with the present invention a methyl group may be attached to the terminal hydroxyl group of the polyoxyethylene/polyoxypropylene moiety (i.e., the terminal hydroxyl group may be blocked). Such blocking of the terminal hydroxyl group may facilitate synthesis. This is particularly useful for compounds not made through ethoxylation or propoxylation, such as compounds made in accordance with Scheme 1 or Scheme 2 above. Structures with a methyl-blocked polyoxyethylene/polyoxypropylene moiety are usually referred as mPEG derivatives, as illustrated by the following exemplary structures:

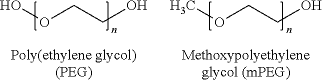

As will be clear to a person skilled in the art, also the synthesis of polyoxypropylene ethers usually yields products with a wide polydispersity of polyoxypropylene repeat unit lengths. Hence, when the number of polyoxypropylene repeat units is indicated for the polyoxypropylene ethers in accordance with the present invention, this number refers to the average polyoxypropylene repeat unit length. As described for polyoxyethylene ethers above, the average polyoxypropylene repeat unit length refers to the average number of polyoxypropylene repeat units of all polyoxypropylene ether molecules of a sample.

The polyoxypropylene ether detergents used in accordance with the present invention have an average number of polyoxypropylene repeat units of 2 to 100, 2 to 50, 2 to 20, or 5 to 15, or 9 or 10. Preferably, the average number of polyoxypropylene repeat units is 5 to 15, more preferably 9 or 10, and most preferably 10.

A method for inactivating a virus having a lipid envelope according to the present invention comprises the step of adding a detergent of the invention to a liquid to prepare a mixture of said detergent and said liquid and a step of incubating said mixture to inactivate said virus. As described above, the term “inactivating a virus having a lipid envelope” as used herein refers reducing the concentration of infectious virus particles in a solution. In the method for inactivating a virus having a lipid envelope according to the present invention, it is preferable that the method achieves a 1 Log 10 reduction value (LRV) for at least one virus of at least 1, or at least 2, or at least 3, or at least 4, or at least 5, or at least 6, or at least 7, or at least 8, and most preferably at least a 4 Log 10 reduction value (LRV) for at least one virus. Of course, it is evident for a skilled person in the art that any Log 10 reduction value (LRV) for at least one virus is beneficial, because it improves the safety of e.g. the biopharmaceutical production process. The LRVs referred to in accordance with the invention are LRVs of an enveloped virus.

It is to be understood that although the methods of the present invention are generally for inactivating lipid-enveloped viruses, the detergents in accordance with the present invention may also inactivate non-enveloped viruses, for example if such non-enveloped viruses acquire a lipid envelope at some stage during their replication cycle. Thus, the term “inactivating a virus having a lipid envelope” is not meant to exclude the possibility that in the methods of the invention, non-enveloped viruses can also be inactivated in addition to viruses having a lipid envelope.

In the method for inactivating a virus having a lipid envelope according to the present invention a detergent of the invention is added to a liquid to prepare a mixture of said detergent and said liquid, and said mixture is incubated to inactivate said virus. It is to be understood that said liquid of the method of the present invention may be any kind of liquid or a mixture of several liquids, including a solution, a suspension, or a mixture of several suspensions and/or solutions. For example, and without limitation thereto, said liquid in accordance with the present invention may be blood or may contain blood or a blood fraction, may be plasma or may contain plasma or a plasma fraction, may be serum or may contain serum or a serum fraction, may be cell culture medium or may contain cell culture medium, may be a buffer or may contain a buffer. The liquid may also be a process intermediate, e.g. a process intermediate in the preparation of a biopharmaceutical drug.

The liquid in accordance with the present invention may contain a virus having a lipid envelope, or it may be suspected to contain a virus having a lipid envelope (e.g. in case it is blood or contains blood or a blood fraction, plasma or contains plasma or a plasma fraction, serum or contains serum or a serum fraction, or if it contains a biopharmaceutical drug produced in cell culture). In a preferred embodiment of the present invention in accordance with all other embodiments of the invention, said liquid in accordance with the present invention contains virus having a lipid envelope. The origin of said virus having a lipid envelope in said liquid in accordance with the present invention is not particularly limited. For example, the virus may originate from human blood, human plasma or human serum that may be used to prepare the liquid in accordance with the present invention, or from cell culture medium that may be used to prepare the liquid in accordance with the present invention. In particular, if the virus originates from cell culture medium that is used to prepare the liquid in accordance with the present invention, the virus may originate from animal-derived components of said cell culture medium, such as bovine serum albumin.

In the method for inactivating a virus having a lipid envelope according to the present invention, a detergent is added to a liquid to prepare a mixture of said detergent and said liquid. Said detergent is a polyoxyethylene or polyoxypropylene ether of the invention.

In the methods for inactivating a virus having a lipid envelope according to the present invention, a detergent is added to a liquid to prepare a mixture of said detergent and said liquid. In one embodiment of the invention, the polyoxyethylene or polyoxypropylene ether detergents are added to the liquid to yield a final concentration of about 0.03% (w/w) to 10% (w/w), preferably about 0.05% (w/w) to 10% (w/w), more preferably 0.1% (w/w) to 10% (w/w), even more preferably about 0.5% (w/w) to 5% (w/w), most preferably about 0.5% (w/w) to 2% (w/w) polyoxyethylene or polyoxypropylene ether detergent in the liquid.

In a preferred embodiment of the methods for inactivating a virus having a lipid envelope of the present invention, the polyoxyethylene or polyoxypropylene ethers that are used in the methods are suitable for the inactivation of said virus having a lipid envelope. Inactivation as used herein refers to disrupting the ability of the lipid-enveloped virus to infect cells. As will be clear to a person skilled in the art, the ability of a lipid-enveloped virus to infect cells, i.e. the infectivity of a lipid-enveloped virus, is typically assessed by determining the number of infectious virus particles in a solution. Exemplary methods for determining the number of infectious virus particles in a solution are described herein.

In one embodiment, in the method for inactivating a virus having a lipid envelope of the present invention, the step of adding a detergent including a non-phenolic detergent of the present invention and a solvent to a liquid is carried out in a way that a solvent/detergent mixture for inactivation of said virus is prepared. Preferably, said solvent is an organic solvent and even more preferably, said solvent is tri-n-butyl phosphate. It is understood that the concentration of the detergent and the type and concentration of solvent can appropriately be chosen by a skilled person, by taking into account, for instance, the potential viruses present in the liquid, the desired LRV, the properties of the biopharmaceutical drug that may be present in the liquid and the characteristics of the manufacturing process of the biopharmaceutical drug that may be present in the liquid (e.g. at which temperature the inactivation will be carried out). Typically, the final concentrations of an organic solvent and a single detergent during the incubation in accordance with the invention is about 0.01% (w/w) to about 5% (w/w) of organic solvent and about 0.05% (w/w) to about 10% (w/w) of detergent, preferably about 0.1% (w/w) to about 5% (w/w) of organic solvent and about 0.1% (w/w) to about 10% (w/w) of detergent, more preferably about 0.1% (w/w) to about 1% (w/w) of organic solvent and about 0.5% (w/w) to about 5% (w/w) of detergent, most preferably about 0.1% (w/w) to about 0.5% (w/w) of organic solvent and about 0.5% (w/w) to about 2% (w/w) of detergent.

In another embodiment, in the method for inactivating a virus having a lipid envelope of the present invention, the step of adding a detergent including a non-phenolic detergent of the present invention (and optionally also a solvent) to a liquid is carried out in a way that a further detergent is added to the liquid. Preferably, said further detergent is polyoxyethylene (80) sorbitan monooleate (also known as, e.g., Polysorbate 80 or TWEEN 80). Preferably, said solvent is an organic solvent and even more preferably, said solvent is tri-n-butyl phosphate. It is understood that the concentration of the detergent in accordance with the present invention, the type and concentration of the further detergent as well as the type and concentration of the solvent can appropriately be chosen by a skilled person, by taking into account, for instance, the potential viruses present in the liquid, the desired LRV, the properties of the biopharmaceutical drug that may be present in the liquid and the characteristics of the manufacturing process of the biopharmaceutical drug that may be present in the liquid (e.g. at which temperature the inactivation will be carried out). Typically, the final concentration of an organic solvent is about 0.01% (w/w) to about 5% (w/w), the final concentration of the detergent in accordance with the present invention is about 0.05% (w/w) to about 10% (w/w), and the final concentration of the further detergent is about 0.01% (w/w) to about 5% (w/w). Preferably, the final concentration of an organic solvent is about 0.1% (w/w) to about 5% (w/w), the final concentration of the detergent in accordance with the present invention is about 0.1% (w/w) to about 10% (w/w), and the final concentration of the further detergent is about 0.1% (w/w) to about 5% (w/w). More preferably, the final concentration of an organic solvent is about 0.1% (w/w) to about 1% (w/w), the final concentration of the detergent in accordance with the present invention is about 0.5% (w/w) to about 5% (w/w), and the final concentration of the further detergent is about 0.1% (w/w) to about 1% (w/w). Most preferably, the final concentration of an organic solvent is about 0.1% (w/w) to about 0.5% (w/w), the final concentration of the detergent in accordance with the present invention is about 0.5% (w/w) to about 2% (w/w), and the final concentration of the further detergent is about 0.1% (w/w) to about 0.5% (w/w).

In another embodiment in accordance with the invention, only one detergent is used. For example, in one embodiment of the method of the invention, in step a) no further detergent other than the detergent of the invention is added. In another embodiment of the method of the invention, in the method no further detergent other than the detergent of the invention is added. In another embodiment in accordance with the invention, in the use of a detergent of the invention in a method for the inactivation of a virus having a lipid envelope, no further detergent other than the detergent of the invention is used. One advantage of these embodiments is that a single detergent can more easily be removed in subsequent (method) steps. For example, a single detergent can be removed more easily as compared to the three components used in a standard solvent/detergent (S/D) treatment, which typically include two detergents and one solvent, in particular an organic solvent. Thus, in another embodiment in accordance with the invention, the composition comprising a detergent of the invention does not comprise any further detergent other than said detergent.

In another embodiment in accordance with the methods for inactivating a virus, no organic solvent is used. For example, in one embodiment of the virus inactivation method of the invention, in step a) no organic solvent is added. In another embodiment in accordance with the invention, in the use of a detergent of the invention in a method for the inactivation of a virus having a lipid envelope, no organic solvent is used. In another embodiment in accordance with the invention, the composition comprising a detergent of the invention does not comprise any organic solvent.

As outlined above, the method for inactivating a virus having a lipid envelope according to the present invention is particularly useful in biopharmaceutical production processes, wherein the absence of active (i.e., infectious) virus from the final product has to be ensured in order to guarantee patient safety. Hence, in one embodiment, in the methods for inactivating a virus having a lipid envelope according to the present invention, a detergent of the invention is added to a liquid that comprises a biological medicinal product or a biopharmaceutical drug, preferably a biopharmaceutical drug. In a preferred embodiment of the present invention, said biopharmaceutical drug is not a viral vaccine. Biopharmaceutical drugs in accordance with the invention are not particularly limited. They include both recombinant biopharmaceutical drugs and biopharmaceutical drugs from other sources such as biopharmaceutical drugs obtained from human plasma. Biopharmaceutical drugs in accordance with the invention include, without limitation, blood factors, immunoglobulins, replacement enzymes, vaccines, gene therapy vectors, growth factors and their receptors. In a preferred embodiment, the biopharmaceutical drug is a therapeutic protein. Preferred blood factors include factor I (fibrinogen), factor II (prothrombin), Tissue factor, factor V, factor VII and factor VIIa, factor VIII, factor IX, factor X, factor XI, factor XII, factor XIII, von Willebrand Factor (VWF), prekallikrein, high-molecular-weight kininogen (HMWK), fibronectin, antithrombin Ill, heparin cofactor II, protein C, protein S, protein Z, plasminogen, alpha 2-antiplasmin, tissue plasminogen activator (tPA), urokinase, plasminogen activator inhibitor-1 (PAI1), and plasminogen activator inhibitor-2 (PAI2). Factor VIII is a particularly preferred blood factor, and recombinant Factor VIII is even more preferred. The blood factors that can be used in accordance with the present invention are meant to include functional polypeptide variants and polynucleotides that encode the blood factors or encode such functional variant polypeptides. Preferred immunoglobulins include immunoglobulins from human plasma, monoclonal antibodies and recombinant antibodies. The biopharmaceutical drugs in accordance with the invention are preferably the respective human or recombinant human proteins or functional variants thereof.

As indicated above, the biopharmaceutical drug in accordance with the present invention may also be a gene therapy vector, including a viral gene therapy vector. As will be clear to a person skilled in the art, the methods for inactivating a virus having a lipid envelope of the present invention generally do not inactivate non-enveloped viruses. Thus, in a preferred embodiment, where the biopharmaceutical drug in accordance with the present invention is a viral gene therapy vector, such viral gene therapy vector is based on a non-enveloped virus. In a preferred embodiment, such viral gene therapy vector is based on adeno-associated virus (AAV).

In accordance with all other embodiments of the present invention, the method for inactivating a virus having a lipid envelope of the present invention may comprise, after the step of incubating said mixture to inactivate said virus, a step of purifying said biopharmaceutical drug. Preferably, said purifying comprises separating said biopharmaceutical drug from said detergent(s). The skilled person will be aware of various methods to separate a biopharmaceutical drug from detergent(s). Such methods can be selected by a person skilled in the art taking into account the properties of the biopharmaceutical drug, the source from which it is obtained (e.g. recombinantly or from other sources such as from human plasma) and the desired biopharmaceutical application (e.g. whether it will be administered subcutaneously or intravenously, etc.). For example, a biopharmaceutical drug may be separated from detergent(s) using chromatography, such as anion exchange chromatography or cation exchange chromatography. In one embodiment, said purifying from said detergent(s) comprises more than one chromatographic purifications.

In accordance with all other embodiments of the present invention, the method for inactivating a virus having a lipid envelope of the present invention may comprise a step of filtering said mixture, preferably with a depth filter. This step of filtering can be carried out before the step of adding a detergent to a liquid to prepare a mixture of said detergent and said liquid. Alternatively, this step of filtering can be carried out between the step of adding a detergent to a liquid to prepare a mixture of said detergent and said liquid and the step of incubating said mixture to inactivate said virus.

In another embodiment in accordance with all other embodiments of the present invention, in the methods for inactivating a virus having a lipid envelope, the step of incubating said mixture to inactivate said virus can be carried out in a way that said mixture is incubated for at least 10 min, for at least 30 min, for at least 1 hour, for at least 2 hours, for at least 4 hours, for at least 12 hours or for at least 24 hours.

In another embodiment in accordance with all other embodiments of the present invention, in the step of incubating said mixture to inactivate said virus, said mixture is incubated at a low temperature, such as a temperature between 0° C. and 15° C., between 0° C. and 10° C., between 0° C. and 8° C., between 0° C. and 6° C., between 0° C. and 4° C., or between 0° C. and 2° C., preferably between 0° C. and 10° C. In an alternative embodiment in accordance with all other embodiments of the present invention, in the step of incubating said mixture to inactivate said virus, said mixture is incubated at or close to room temperature, such as a temperature between 16° C. and 25° C., between 18° C. and 24° C., or between 20° C. and 23° C.

It is to be understood that the way in which the steps of the method of the present invention are to be carried out is not particularly limited. In particular, the method steps may be carried out in a batch-wise fashion. Alternatively, the method steps may also be carried out in a semi-continuous or continuous fashion.

As outlined above, the method for inactivating a virus having a lipid envelope according to the present invention is particularly useful in biopharmaceutical production processes. Thus, the present invention also relates to a method for preparing a biopharmaceutical drug, said method comprising the method steps of the method for inactivating a virus having a lipid envelope in accordance with the present invention and any embodiments thereof. Preferably, the method for preparing a biopharmaceutical drug in accordance with the present invention comprises a step of preparing a pharmaceutical formulation comprising said biopharmaceutical drug, which is performed after the steps of the method for inactivating a virus having a lipid envelope according to the present invention. Such pharmaceutical formulation can be prepared in accordance with known standards for the preparation of pharmaceutical formulations. For example, the formulation can be prepared in a way that it can be stored and administered appropriately, e.g. by using pharmaceutically acceptable components such as carriers, excipients or stabilizers. Such pharmaceutically acceptable components are not toxic in the amounts used when administering the pharmaceutical formulation to a patient.

The present inventors have surprisingly found that the detergents according to the present invention are particularly useful for inactivating virus having a lipid envelope. Thus, the present invention also relates to the use of the disclosed polyoxyethylene or polyoxypropylene ether detergents according to the present invention in any method for the inactivation of a virus having a lipid envelope. Preferably, said method for said inactivation of said virus is a method using a solvent/detergent treatment, wherein said solvent/detergent treatment comprises the use of said detergent of the present invention. In another embodiment, said virus inactivation is an inactivation of virus in a liquid comprising a biopharmaceutical drug.

Since the present inventors have surprisingly found that the non-phenolic detergents according to the present invention are particularly useful for inactivating virus having a lipid envelope, the present invention also relates to the polyoxyethylene or polyoxypropylene ether detergents according to the present invention, and to a composition comprising a polyoxyethylene or polyoxypropylene ether detergent according to the present invention. In another embodiment, the composition comprising a polyoxyethylene or polyoxypropylene ether detergent according to the present invention additionally comprises a biopharmaceutical drug and/or an organic solvent and/or a further detergent.

The present invention provides non-phenolic polyoxyethylene or polyoxypropylene ethers. As described above, these polyoxyethylene or polyoxypropylene ethers can be used in the methods for inactivating a virus having a lipid envelope according to the present invention. However, these non-phenolic polyoxyethylene or polyoxypropylene ethers, as well as all other non-phenolic polyoxyethylene or polyoxypropylene ethers in accordance with the present invention, can also be used for various other purposes, e.g. for those in which Triton X-100 is commonly used. For example, the non-phenolic polyoxyethylene or polyoxypropylene ethers in accordance with the present invention can be used in the laboratory. In the laboratory they can be used, e.g., to lyse cells to extract protein or organelles, or to permeabilize the membranes of living cells; to permeabilize unfixed (or lightly fixed) eukaryotic cell membranes; to solubilize membrane proteins in their native state in conjunction with zwitterionic detergents such as CHAPS; as part of the lysis buffer (usually in a 5% solution in alkaline lysis buffer) in DNA extraction; to reduce surface tension of aqueous solutions during immunostaining (usually at a concentration of 0.1-0.5% in TBS or PBS Buffer); to restrict colony expansion in Aspergillus nidulans in microbiology; to decellularize animal-derived tissues; or to remove SDS from SDS-PAGE gels prior to renaturing the proteins within the gel. In another embodiment, the non-phenolic polyoxyethylene or polyoxypropylene ethers in accordance with the present invention can be used in the electronic industry, e.g. as a wetting agent for the slats to improve and speed up some procedures and operations. In another embodiment, the non-phenolic polyoxyethylene or polyoxypropylene ethers in accordance with the present invention can have medical uses, e.g. they can be used as a substitute for the spermicide Nonoxynol 9, or as a pharmaceutical excipient, or as an ingredient in influenza vaccine (Fluzone). In further embodiments, the non-phenolic polyoxyethylene or polyoxypropylene ethers in accordance with the present invention can be used in several types of cleaning compounds, ranging from heavy-duty industrial products to gentle detergents; they can be used as an ingredient in homemade vinyl record cleaning fluids together with distilled water and isopropyl alcohol; they can be used during the cleaning of diamond blades; they can be used in formulations for polymerization of emulsions; they can be used in tires; they can be used in washing and cleaning agents; they can be used in industry as starting chemical for the production of polymers or glues; they can be used in household or industrial cleaners, in paints or coatings, in pulp or paper, on the oilfield, in textiles, in agrochemicals, in metalworking fluids; they can be used for the dispersion of carbon materials for soft composite materials; or they can be used in the plating of metal.

In the following, the present invention will be illustrated by examples, without being limited thereto.

### EXAMPLES

As outlined above, recent ecological studies have raised environmental concerns regarding the use of Triton X-100 in biopharmaceutical production processes. Based on structural considerations, the present inventors have identified candidate detergents as useful alternatives to Triton X-100 which have not been associated with negative environmental impact. In particular, the present inventors have surprisingly identified non-phenolic polyoxyethylene ethers as suitable alternatives to Triton X-100 for inactivating lipid-enveloped viruses by solvent/detergent (S/D) treatment during biopharmaceutical production. In the following experiments, 4-tert-octylbenzyl alcohol polyethoxylate was synthesized, and the suitability of 4-tert-octylbenzyl alcohol polyethoxylate and other polyoxyethylene ethers for S/D treatment as well as single-detergent treatment was tested on various test items.

### Example 1: Synthesis of 4-tert-octylbenzyl Alcohol Polyethoxylate I (Method 1)

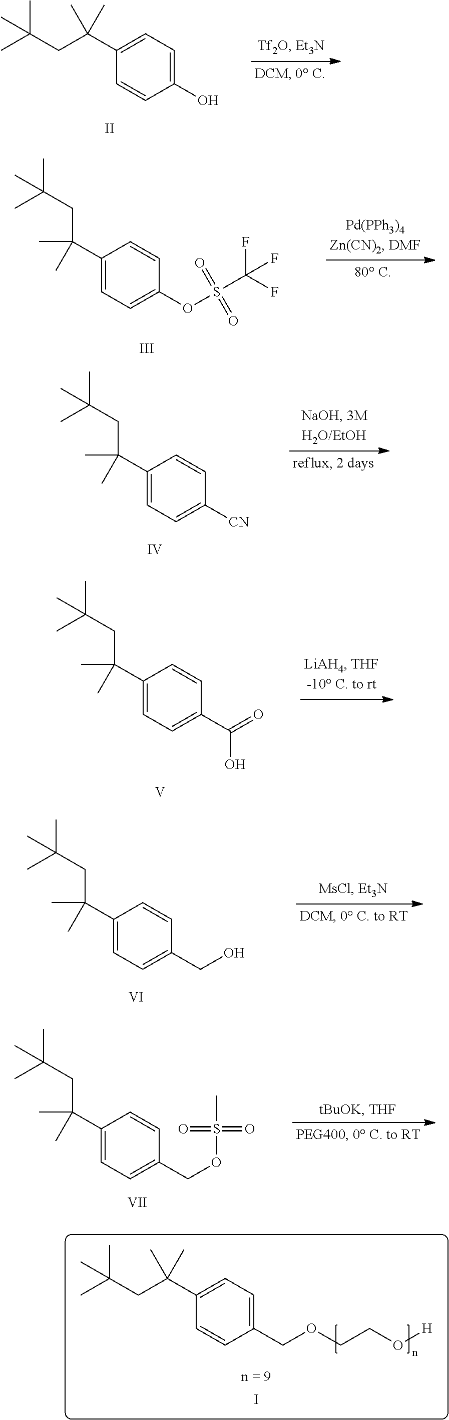

### Synthesis of Intermediate III

Phenol II (170.3 g, 800 mmol) was placed in a 3 neck 2 L flask equipped with an inner thermometer and stirring bar. Anhydrous CH₂Cl₂ (1000 mL) was added to the flask and stirring was started. After dissolution of the starting material the solution was cooled to 0° C. After total dissolution NEt₃ (225 mL, 1.6 mol) was added within 10 min. A solution Tf₂O (256 g, 907 mmol) in CH₂Cl₂ (180 mL) was added at 0° C. to the reaction mixture over 120 min and the reaction was stirred overnight at room temperature. An aqueous saturated NaHCO₃ solution (400 mL) was added and the reaction mixture was extracted. The organic phase was washed repeatedly with water (2×400 mL) and brine (500 mL). The organic phase was then concentrated in vacuo. Toluene (300 mL) was added to the residue and the crude product was concentrated to yield 314 g of crude black liquid. This residue was charged on top of a SiO₂ plug and eluted with petroleum ether/EtOAc (0 to 2%). Concentration of the pure fractions yielded 269.5 g (Yield: 99.6%) of a clear colorless oil. R_{f}=0.74 (petroleum ether/EtOAc 5%).

### Synthesis of Intermediate IV

Triflate III (269 g, 795 mmol) was dissolved in anhydrous and degassed DMF (1.3 L) and Zn(CN)₂ (95.3 g, 795 mmol) and Pd(PPhs)₄ (25 g, 21.5 mmol) were added sequentially. The reaction mixture was warmed to 80° C. for 3 hrs followed by removal of the DMF under vacuum. Toluene (300 mL) was added to the residue and the crude product was concentrated to yield 432 g of black residue. This crude product was charged on top of a SiO₂ plug and eluted with petroleum ether/EtOAc (0 to 10%). Concentration of the pure fractions yielded 128.1 g (Yield: 74.8%) of a clear colorless oil. R_{f}=0.23 (petroleum ether/EtOAc 2%).

### Synthesis of Intermediate V

Nitrile IV (127.6 g. 592.5 mmol) dissolved in MeOH (500 mL), aqueous NaOH 4M (750 mL) was added and the mixture was brought to reflux and kept at this temperature (80° C.) overnight. An additional aqueous NaOH 10M (150 mL) was added to the warm mixture and the solution was heated further for 20 hrs. After cooling to ambient temperature the content of the reaction vessel was transferred into a large Beaker and cooled in an ice bath. Aqueous HCl 4M (1.1 L) was added within 30 min, at this point the pH was acidic as indicated by pH paper and a white solid had precipitated. The precipitate was filtered and rinsed with water (500 mL). The wet cake was transferred to a 2 L flask and dried under vacuum for 3 days to yield 127.5 g (Yield: 91.9%) of a white powder. R_{f}=0.42 (petroleum ether/EtOAc 2:1).

### Synthesis of Intermediate VI

Carboxylic acid V (127 g, 542 mmol) was suspended in dried mTHF (1.2 L) and cooled to −10° C. A solution of LiAlH₄ in THF (1 M, 575 mL, 575 mmol) was added over 60 min, then the reaction was warmed slowly to ambient temperature and further stirred overnight. The content of the reaction vessel was transferred into a large Beaker and the excess of hydride was carefully quenched with ice (15 g). Aqueous HCl 3M (500 mL) was added within 20 min, at this point the pH was acidic as indicated by pH paper. EtOAc (300 mL) was added to the crude mixture and the 2 phases were vigorously agitated. The aqueous phase was back extracted twice with EtOAc (300 mL and 500 mL). The combined organic phases were washed successively with an aqueous saturated NaHCO₃ solution (300 mL), water (300 mL) and brine (500 mL). The organic phase was then concentrated in vacuo. Toluene (300 mL) was added to the residue and the crude product was concentrated to yield 123.3 g of a clear yellowish oil. This residue was charged on top of a SiO₂ plug and eluted with petroleum ether/EtOAc (0 to 15%). Concentration of the pure fractions yielded 85.3 g (Yield: 71.4%) of an amorphous white solid. R_{f}=0.37 (petroleum ether/EtOAc 4:1).

### Synthesis of Intermediate VII

Benzyl alcohol VI (84.8 g, 385 mmol) was dissolved in anhydrous CH₂Cl₂ (1 L) and cooled in an ice/water bath. NEt₃ (110 mL, 770 mmol) was added followed by the slow addition of a solution of MsCl (45 mL, 577 mmol) in anhydrous CH₂Cl₂ (25 mL) over 60 min. The reaction warmed slowly to ambient temperature and was further stirred overnight. An aqueous saturated NaHCO₃ solution (420 mL) was added and the reaction mixture was agitated vigorously. The organic phase was washed repeatedly with water (2×500 mL) and brine (300 mL). The organic phase was then concentrated in vacuo. Toluene (200 mL) and CH₂Cl₂ (100 mL) were added to the residue and the crude product was concentrated to yield 90 g (Yield: 78.4%) of an orange semi solid. R_{f}=0.71 (petroleum ether/EtOAc 4:1).

### Synthesis of I

PEG400 (360 g, 900 mmol) was dissolved in anhydrous THF (1 L) and tBuOK (90 g, 802 mmol) was added at ambient temperature portion wise over 15 min and the mixture was stirred 60 min at ambient temperature and cooled in an ice bath. In the meantime, Mesylate VII (89.5 g, 300 mmol) was suspended in THF (300 mL) and the milky orange solution was added to the cooled deprotonated PEG400 solution over 20 min. The reaction was warmed slowly to ambient temperature and further stirred overnight. Ice (500 g) was added as well as aqueous HCl 1M (820 mL). The THF was removed under vacuum and EtOAc (1 L) was added. The phases were agitated and the organic phase was washed successively with water (2×500 mL). Each aqueous phase was back extracted with EtOAc (300 mL). The combined organic phases were washed with water (500 ml) and concentrated in vacuuo. Toluene (250 mL) was added to the residue and the crude product was concentrated to yield 125.2 g of a clear yellowish oil. This residue was charged on top of a SiO₂ plug and eluted with CH₂Cl₂/MeOH (0 to 8%). Concentration of the pure fractions yielded 107.5 g (Yield: 59.5%) of a light brown clear oil. R_{f}=0.37-0.22 (CH₂Cl₂/MeOH 20:1). MS (ESI): m/z=[M+H]⁺=573.5, 617.5 (100%), 661.6; [M+Ac]-=631.4, 675.4 (100%), 719.5. ¹H-NMR (600 MHz, CDCl₃): δ=7.33 (d, J=8.3 Hz, 2H), 7.23 (d, J=8.3 Hz, 2H), 4.52 (s, 2H), 3.72-3.58 (m, 33H), 2.54 (br s, 1H), 1.72 (s, 2H), 1.34 (s, 6H), 0.70 (s, 9H). ¹³C-NMR (150 MHz, CDCl₃): δ=149.7, 135.1, 127.4 (2C), 126.2 (2C), 73.2, 72.6, 70.7 (m), 70.5, 69.4, 61.9, 57.0, 38.6, 32.5, 31.9 (3C), 31.6 (2C).

### Example 2a: Synthesis of 4-tert-octylbenzyl Alcohol Polyethoxylate I (Method 2)

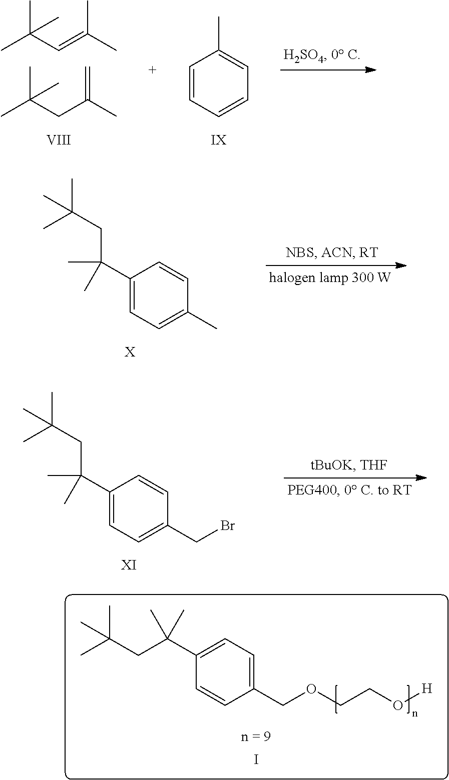

Toluene (35 mL, 328 mmol) and concentrated H₂SO₄ (10 mL) were cooled to 0° C. in a round bottom flask. A mixture of toluene (27 mL, 256 mmol) and diisobutylene (as a mixture 3:1 2,4,4-trimethyl-1-pentene+2,4,4-trimethyl-2-pentene, 10 mL, 64 mmol) was added slowly to the reaction mixture over 2 hrs. The reaction was further stirred at 0° C. for 2 hrs. Water (100 mL) was added. After phase separation the organic phase was washed with an aqueous saturated NaHCO₃ solution (100 mL), dried over MgSO₄ and concentrated to yield 14 g of a clear colorless oil. The oil was placed in a 100 mL round bottom flask and distillated over a short path distillation apparatus (1-10⁻¹ mbar). The fraction (8.1 g, Yield 61.9%) that distillated between 62 and 70° C. was collected. R_{f}=0.65 (Petrol ether 40-60 100%).

p-Substituted toluene X (500 mg, 2.45 mmol) was dissolved in acetonitrile (ACN) (5 mL). N-bromosuccinimide (NBS) (460 mg, 2.57 mmol) was added and after dissolution, the Duran 25 mL round bottom flask was placed 15 cm away from the halogen lamp (300 W). After 60 min of irradiation (Reaction temperature=45° C.) the solvent was removed. Petrol ether 40-60 (25 mL) was added and a solid precipitated. The liquid phase was washed with water (2×20 mL), dried over MgSO₄ and concentrated to yield 540 mg (Yield: 77.9%) of a crude yellowish oil. R_{f}=0.43 (Petrol ether 40-60 100%).

PEG400 (2.25 g, 5.61 mmol) was dissolved in anhydrous THF (5 mL) at ambient temperature. tBuOK (420 mg, 3.74 mmol) was added portion wise over 1 min and the mixture was stirred 90 min and then cooled to 0° C. in an ice/water bath. In the meantime, the benzyl bromide intermediate XI (530 mg, 1.87 mmol) was suspended in THF (2 mL) and the solution was added to the cooled deprotonated PEG400 solution. The reaction was warmed slowly to ambient temperature and further stirred overnight. HCl (1M, 20 mL) was added to the reaction mixture as well as EtOAc (50 mL) and water (20 mL). The solution was transferred into a separatory funnel and extracted vigorously. After phase separation the organic phase was washed successively with water (5×15 mL) and finally dried over MgSO₄ to yield 0.8 g of crude oily residue. This residue was charged on top of a SiO₂ column and eluted with CH₂Cl₂/MeOH (0 to 8%). Concentration of the pure fractions yielded 588 mg (Yield: 52.2%) of a clear yellowish oil. R_{f}=0.37-0.22 (CH₂Cl₂/MeOH 20:1).

### Example 2b: Synthesis of 4-tert-octylbenzyl Alcohol Polyethoxylate I (Method 2 Variants)

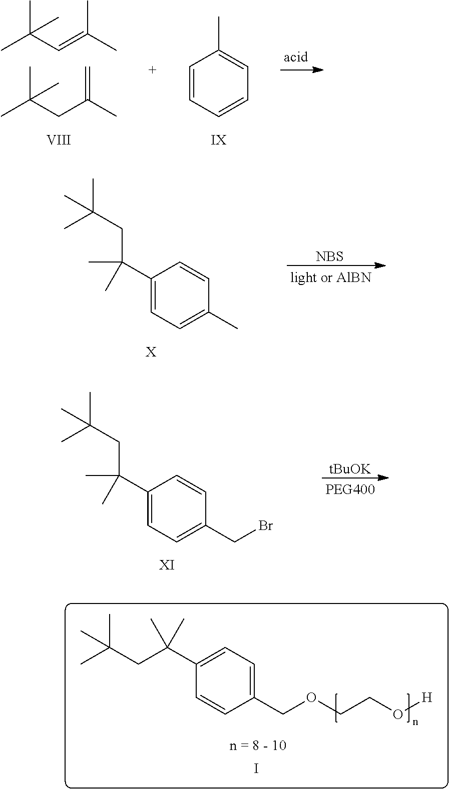

### Step 1:

This step was carried out as follows:

Toluene (750 mL, 7.04 mol) and concentrated H₂SO₄ (20 mL, 0.375 mol) were cooled to 0° C. in a round bottom flask. A mixture of toluene (250 mL, 2.35 mol) and diisobutylene (as a mixture 3:1 2,4,4-trimethyl-1-pentene+2,4,4-trimethyl-2-pentene, 200 mL, 1.28 mol) was added slowly to the reaction mixture over 90 min. The reaction was further stirred at 0° C. and warmed to ambient temperature overnight. Ice (400 g) was added and the mixture was transferred to a separatory funnel. After phase separation the organic phase was washed successively with an aqueous saturated NaHCO₃ solution (300 mL) and water (2×250 mL). The organic phase was concentrated to yield 199.1 g of a clear colorless oil. The oil was placed in a 500 mL round bottom flask and distilled over a short path distillation apparatus (20 mbar). The fractions that distilled between 138° C. and 161° C. were collected (134.1 g, Yield 51.4%). R_{f}=0.65 (Petrol ether 40-60 100%). ¹H-NMR (600 MHz, CDCl₃): δ=7.28 (d, J=8.2 Hz, 2H), 7.10 (d, J=8.2 Hz, 2H), 2.34 (s, 3H), 1.75 (s, 2H), 1.38 (s, 6H), 0.75 (s, 9H). ¹³C-NMR (150 MHz, CDCl₃): δ=147.3, 134.7, 128.6 (2C), 126.1 (2C), 57.1, 38.4, 32.5, 31.9 (3C), 31.7 (2C), 21.0.

Alternatively, this step was carried out as follows:

Toluene (400 mL, 3.83 mol) and nonafluoro-1-butanesulfonic acid (4 mL, 24 mmol) were stirred at ambient temperature in a round bottom flask. A mixture of toluene (200 mL, 1.92 mol) and diisobutylene (as a mixture 3:1 2,4,4-trimethyl-1-pentene+2,4,4-trimethyl-2-pentene, 100 mL, 0.64 mol) was added slowly to the reaction mixture over 60 min. The reaction was further stirred at ambient temperature overnight. Aqueous saturated NaHCO₃ solution (200 mL) was added and the mixture was stirred for 20 min. The mixture was transferred to a separatory funnel and the aqueous phase was discarded. The organic phase was washed successively with water (3×300 mL). The organic phase was concentrated to yield 132.5 g of a clear colorless oil. The oil was placed in a 500 mL round bottom flask and distillated over a short path distillation apparatus (26-16 mbar). The fractions that distilled between 115° C. and 135° C. were collected (80.5 g, Yield 62%). R_{f}=0.65 (Petrol ether 40-60 100%). ¹H-NMR (600 MHz, CDCl₃): δ=7.28 (d, J=8.2 Hz, 2H), 7.10 (d, J=8.2 Hz, 2H), 2.34 (s, 3H), 1.75 (s, 2H), 1.38 (s, 6H), 0.75 (s, 9H). ¹³C-NMR (150 MHz, CDCl₃): δ=147.3, 134.7, 128.6 (2C), 126.1 (2C), 57.1, 38.4, 32.5, 31.9 (3C), 31.7 (2C), 21.0.

### Step 2:

This step was carried out as follows:
p-Substituted toluene X (63.6 g, 311 mmol) was dissolved in Acetonitrile (ACN) (650 mL). N-bromosuccinimide (NBS) (58.2 g, 327 mmol) was added and after dissolution, the Duran 2 L round bottom flask was placed 5-25 cm away from the halogen lamp (300 W) while stirring (350 rpm). After 6 hrs of irradiation (Reaction temperature=up to 46° C.) the solvent was removed under vacuo. Petrol ether 40-60 (450 mL) was added and a dark solid precipitated. The liquid phase was concentrated to yield a dark brown residue (75 g). This residue was charged on top of a SiO₂ plug and eluted with petrol ether 40-60 (100%). Concentration of the pure fractions yielded 43.8 g (Yield: 49.7%) of an orange clear oil. R_{f}=0.43 (Petrol ether 40-60 100%). ¹H-NMR (600 MHz, CDCl₃): δ=7.34 (d, J=8.4 Hz, 2H), 7.30 (d, J=8.4 Hz, 2H), 4.50 (s, 2H), 1.74 (s, 2H), 1.36 (s, 6H), 0.72 (s, 9H). ¹³C-NMR (150 MHz, CDCl₃): δ=150.9, 134.8, 128.6 (2C), 126.7 (2C), 57.0, 38.7, 33.9, 32.5, 31.9 (3C), 31.6 (2C).

Alternatively, this step was carried out as follows:
p-Substituted toluene X (85.2 g, 417 mmol) was dissolved in Trifluorotoluene (830 mL). Other solvents such as esters or alkanes (EtOAc and hexane) are also effective. N-bromosuccinimide (NBS) (74.2 g, 417 mmol) was added as well as AIBN (Azobis(isobutyronitrile) (3.4 g, 21 mmol) while stirring (450 rpm). The mixture was heated to 80° C. for 5 hours. The solvent was removed under vacuo. Petrol ether 40-60 (350 mL) was added and a white solid precipitated. The liquid phase was concentrated to yield a clear orange residue (108 g). This residue was charged on top of a SiO₂ plug and eluted with petrol ether 40-60 (100%). Concentration of the pure fractions yielded 64.1 g (Yield: 54.3%) of a clear almost colorless oil that crystallized to form colorless needles, indicating high purity of the product. R_{f}=0.43 (Petrol ether 40-60 100%). ¹H-NMR (600 MHz, CDCl₃): δ=7.34 (d, J=8.4 Hz, 2H), 7.30 (d, J=8.4 Hz, 2H), 4.50 (s, 2H), 1.74 (s, 2H), 1.36 (s, 6H), 0.72 (s, 9H). ¹³C-NMR (150 MHz, CDCl₃): δ=150.9, 134.8, 128.6 (2C), 126.7 (2C), 57.0, 38.7, 33.9, 32.5, 31.9 (3C), 31.6 (2C). It is expected that the use of AIBN (azobis(isobutyronitrile) as a radical initiator contributes to the high purity of the product which is obtained in this reaction step.

### Step 3:

This step was carried out as follows:
PEG400 (184.1 g, 460 mmol) was dissolved in anhydrous THF (550 mL) at ambient temperature. tBuOK (27.2 g, 245 mmol) was added portionwise over 15 min and the mixture was stirred 90 min. In the meantime, the benzyl bromide intermediate XI (43.5 g, 153 mmol) was suspended in THF (300 mL) and the solution was added to the cooled deprotonated PEG400 solution. The reaction was stirred overnight at ambient temperature. HCl (1M, 270 mL) was added to the reaction mixture. Volatiles were removed and EtOAc (600 mL) was added. The solution was transferred into a separatory funnel and extracted vigorously. After phase separation the aqueous phase was further extracted with EtOAc (300 mL). The combined organic phases were washed successively with water/brine (1:1, 3×300 mL) and finally concentrated to yield 90.4 g of crude orange oily residue. This residue was charged on top of a SiO₂ column and eluted with CH₂Cl₂/MeOH (0 to 8%). Concentration of the pure fractions yielded 82.2 g (Yield: 89.0%) of a clear light brown oil. R_{f}=0.37-0.22 (CH₂Cl₂/MeOH 20:1). MS (ESI): m/z=[M+H]⁺=573.5, 617.5 (100%), 661.6; [M+Ac]⁻=631.4, 675.4 (100%), 719.5. ¹H-NMR (600 MHz, CDCl₃): δ=7.33 (d, J=8.3 Hz, 2H), 7.23 (d, J=8.3 Hz, 2H), 4.52 (s, 2H), 3.72-3.58 (m, 33H), 2.54 (br s, 1H), 1.72 (s, 2H), 1.34 (s, 6H), 0.70 (s, 9H). ¹³C-NMR (150 MHz, CDCl₃): δ=149.7, 135.1, 127.4 (2C), 126.2 (2C), 73.2, 72.6, 70.7 (m), 70.5, 69.4, 61.9, 57.0, 38.6, 32.5, 31.9 (3C), 31.6 (2C).

Alternatively, this step was carried out as follows:
PEG400 (475 g, 1.19 mol) was dissolved in TBME (methyl-tert-butylether) (1.0 L) at ambient temperature. tBuOK (43.2 g, 385 mmol) was added portion wise over 20 min and the mixture was stirred 90 min. In the meantime, the benzyl bromide intermediate XI (84 g, 297 mmol) was suspended in TBME (250 mL) and the solution was added to the deprotonated PEG400 solution at ambient temperature. The reaction was stirred 3 hrs at ambient temperature. Ice (200 g) and HCl (1M, 400 mL) were added to the reaction mixture. The solution was transferred into a separatory funnel and EtOAc (1 L) and water (500 mL) were added and extracted vigorously. After phase separation the organic phase was washed successively with water/brine (1:1, 3×500 mL) and finally concentrated to yield 165 g of crude orange oily residue. This residue was charged on top of a SiO₂ column and eluted with CH₂Cl₂/MeOH (0 to 10%). Concentration of the pure fractions yielded 151.7 g (Yield: 84.9%) of a clear light brown oil. R_{f}=0.37-0.22 (CH₂Cl₂/MeOH 20:1). MS (ESI): m/z=[M+H]⁺=573.5, 617.5 (100%), 661.6; [M+Ac]⁻=631.4, 675.4 (100%), 719.5. ¹H-NMR (600 MHz, CDCl₃): δ=7.33 (d, J=8.3 Hz, 2H), 7.23 (d, J=8.3 Hz, 2H), 4.52 (s, 2H), 3.72-3.58 (m, 33H), 2.54 (br s, 1H), 1.72 (s, 2H), 1.34 (s, 6H), 0.70 (s, 9H). ¹³C-NMR (150 MHz, CDCl₃): δ=149.7, 135.1, 127.4 (2C), 126.2 (2C), 73.2, 72.6, 70.7 (m), 70.5, 69.4, 61.9, 57.0, 38.6, 32.5, 31.9 (3C), 31.6 (2C). It is expected that the use of TBME (methyl-tert-butylether) as a solvent, the moderate reaction time of 3 hours (which is expected to minimize reaction side products), the larger manufacturing scale and the purity of the starting material (i.e. the benzyl bromide intermediate XI) all contribute to the high yield observed in this reaction step.

### Example 3—Improved Synthesis and Improved Purification of 4-tert-octylbenzyl Alcohol Polyethoxylate

### Step 1 and Step 2

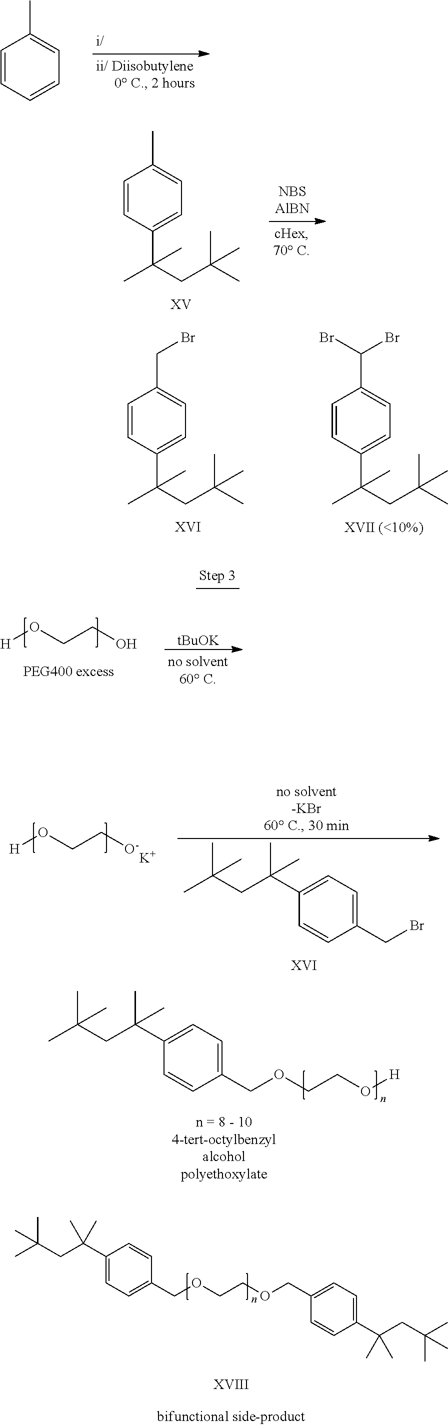

### 1 Material

All the syntheses were carried out with commercially available reagents and solvents (Merck, Karl Roth, TCI, WVR) used without further purification.

### 2 Synthesis and Analytic Methods

¹H and ¹³C-NMR spectra were recorded on a Bruker AV600 spectrometer at room temperature using standard pulse programs. Chemical shifts (6) are quoted in parts per million (ppm) and referenced to the appropriate residual solvent signal. Coupling constants (J) are reported to the nearest 0.1 Hz. LC-Mass spectra (m/z) were run on a Shimadzu LC-MS 2020, HRMS (High resolution mass) analysis was performed on MicroMass TOF spectrometer from Waters (ESI+). Flash chromatography was performed on silica gel (Merck Kieselgel 60, 0.040-0.063 mm). Thin Layer Chromatography (TLC) was performed using TLC Silica Gel 60 F24 (aluminium sheets from Merck). Analytical HPLC chromatograms were run in an Agilent System equipped with DAD and/or ELSD detector. Melting points were measured on a DSC instrument (DSC 2000 from TA Instruments) or on a microscope Reichert Thermovar. FT-IR (ATR) was measured on a Bruker tensor 27 spectrometer equipped with an ATR BioATR cell II using neat samples (for liquid and oily compounds) or using dichloromethane dissolved solution samples (for solid samples) measured after full evaporation of the volatiles. Calculations of molecule lengths were performed using the Software Chem3D V15.1.

### 3 Synthetic Procedures

### 3.1 Synthesis of 1-methyl-4-(2,4,4-trimethylpentan-2-yl)benzene (XV) (Step (1))

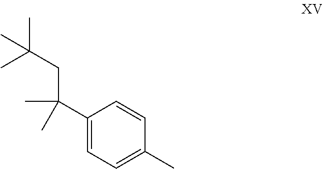

At ambient temperature and under stirring, the reaction vessel was charged with toluene (V=600 mL, 4 vol.) and nonafluoro-1-butanesulfonic acid (V=4.8 mL) was added in one portion. The solution was cooled to 0° C. and a mixture of diisobutylene (V=150 mL) premixed in toluene (V=300 mL) was added slowly over 40-100 min to the reaction mixture using a dropping funnel, while cooling the reaction mixture in the ice/water bath. Then the reaction mixture was stirred 120 min at ambient temperature before aq. saturated NaHCO₃ (V=300 mL) was added in one portion to quench the reaction. The reaction mixture was stirred for 60 min and the content of the flask was transferred to a separatory funnel. The aqueous phase was removed after phase separation and the organic phase was washed with water (2×V=500 mL). The organic phase was concentrated thoroughly in vacuo to yield the crude product as a light brown oily residue (195.5 g, quantitative yield). R_{f}=0.65 (Petrol ether 40-60 100%). ¹H-NMR (600 MHz, CDCl₃): δ=7.28 (d, J=8.2 Hz, 2H), 7.10 (d, J=8.2 Hz, 2H), 2.34 (s, 3H), 1.75 (s, 2H), 1.38 (s, 6H), 0.75 (s, 9H). ¹³C-NMR (150 MHz, CDCl₃): δ=147.3, 134.7, 128.6 (2C), 126.1 (2C), 57.1, 38.4, 32.5, 31.9 (3C), 31.7 (2C), 21.0. IR (cm⁻¹): 2953, 2906, 2867, 1513, 1469, 1364, 1251, 1020, 815, 649, 489.

Further, it was found that triflic acid, which is cheaper and easier to handle (less viscous and easier to dissolve) than nonafluoro-1-butanesulfonic acid, can advantageously be used instead of nonafluoro-1-butanesulfonic acid.

### 3.2 Synthesis of 1-(bromomethyl)-4-(2,4,4-trimethylpentan-2-yl)benzene (XVI) (Step (2))

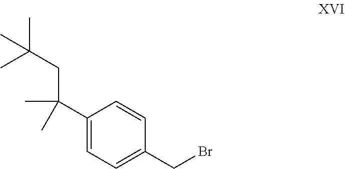 [SMILES: CC(C)(C)CC(C)(C)c1ccc(CBr)cc1]

para-Substituted toluene XV (195 g, 954 mmol) was dissolved in cyclohexane (1560 mL, 8 vol.) and NBS (170 g, 954 mmol) and AIBN (0.8 g, 4.8 mmol) are added while stirring. The mixture was heated to 70° C. until completion and cooled down to ambient temperature. The solid was filtered off and the cake was rinsed with cHex (100 mL). The filtrate was transferred into a separatory funnel and the organic phase was washed with water (2×500 mL). The organic phase was concentrated in vacuo to yield a crude residue as a brown clear oil. IPA (2 mL/g of residue) was added to the crude and the solution cooled to −20° C. After 1 hr at −20° C. a few seed crystals (10-50 mg) were added. The flask was left at −20° C. overnight. The solid was filtered off, rinsed with cold IPA (50 mL) to yield an off-white solid and the corresponding mother liquor. This solid was re-dissolved in IPA (2 mL/g solid) and water (0.2 mL/g solid), using the water bath of the rotavapor (45° C., 5 min). The solution was cooled to +2-8° C. After 1 hr at +2-8° C. a few seed crystals (10-50 mg) were added and the flask was left at +2-8° C. overnight and further cooled to −20° C. for 4 hours. Repeat crystallization if necessary. The precipitate was filtered off, rinsed with cold IPA (50 mL) to yield colorless needles. The solid was dried in the vacuum oven (30° C., <15 mbar, 24 hrs followed by 20° C., <15 mbar, 24 hrs) to yield 50-80 g (20-30% yield over 2 steps). R_{f}=0.43 (Petrol ether 40-60 100%). m.p. (DSC)=53° C. ¹H-NMR (600 MHz, CDCl₃): δ=7.34 (d, J=8.4 Hz, 2H), 7.30 (d, J=8.4 Hz, 2H), 4.50 (s, 2H), 1.73 (s, 2H), 1.36 (s, 6H), 0.72 (s, 9H). ¹³C-NMR (150 MHz, CDCl₃): δ=150.9, 134.8, 128.7 (2C), 126.7 (2C), 57.0, 38.7, 33.9, 32.5, 31.9 (3C), 31.6 (2C). IR (cm⁻¹): 2953, 2899, 2868, 1511, 1470, 1366, 1252, 1230, 1204, 1095, 830, 648, 628, 606, 573, 454.

Further, it was also found that it is generally possible to use acetonitrile instead of cyclohexane as the solvent for this step. Acetonitrile can advantageously be used both as the solvent for the synthesis and as the solvent for the crystallization. In addition, using acetonitrile allows to reduce the formation of di bromated side product described in the following section.

### 3.3 Synthesis of the Side Product XVII for Comparison

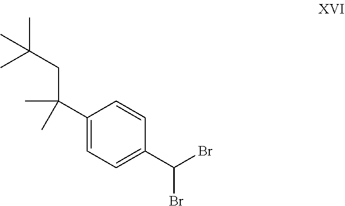 [SMILES: CC(C)(C)CC(C)(C)c1ccc(C(Br)Br)cc1]

para-Substituted benzyl bromide XVI (502 mg, 1.77 mmol) was dissolved in cyclohexane (6 mL). NBS (474 mg, 417 mmol) was added as well as AIBN (16 mg, 0.09 mmol) while stirring (400 rpm). The mixture was heated to 80° C. for 4 hours. The reaction mixture was cooled to ambient temperature and 20 mL cyclohexane was added. The content of the flask was transferred into a separatory funnel and the organic phase was washed with water (2×5 mL) followed by brine (5 mL). The solvent was removed under vacuo to yield a yellowish residue (720 mg). This residue was charged on top of a SiO₂ column and eluted with hexane (100%). Concentration of the pure fractions yielded 358 mg (Yield: 56%) of a clear colorless viscous oil. R_{f}=0.47 (hexane). ¹H-NMR (600 MHz, CDCl₃): δ=7.47 (d, J=8.4 Hz, 2H), 7.36 (d, J=8.4 Hz, 2H), 6.65 (s, 1H), 1.74 (s, 2H), 1.36 (s, 6H), 0.72 (s, 9H). ¹³C-NMR (150 MHz, CDCl₃): δ=152.6, 139.1, 126.5 (2C), 126.1 (2C), 57.0, 41.3, 38.9, 32.5, 31.9 (3C), 31.5 (2C). IR (cm⁻¹): 2955, 2898, 2871, 1607, 1469, 1411, 1365, 1251, 1143, 1095, 1017, 836, 746, 667.

### 3.4 Synthesis of 4-tert-octylbenzyl Alcohol Polyethoxylate from XVI (Step (3))

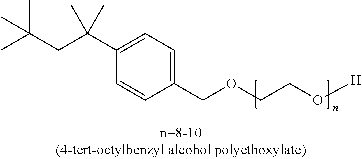

A reaction flask was filled with PEG400 (700 g, 1.77 mol) and heated to 60° C. before tBuOK (47.5 g, 423 mmol) was added portion wise under stirring. After addition, the reaction mixture was heated 30 min at 60° C. The solid crystalline benzyl bromide intermediate XVI (100 g) was added in one portion to the deprotonated PEG. After 30 min, the reaction mixture was allowed to cool down to ambient temperature and water (1.5 L) was added. Using 1 M HCl aq. solution, the pH was set to 6-7. Optionally, if the final product needs to be almost colorless: Sodium hypochlorite (5% active Cl solution, 10-15 mL) was added dropwise to the reaction vessel. The content of the vessel was transferred to a separatory funnel. Water (0.5 L) and EtOAc (2 L) were added to the funnel and the phases were shaken vigorously. After phase separation the aqueous phase was removed. Water/brine (20:1) (1 L) was added and the phases were shaken vigorously, after phase separation the aqueous phase was removed. Repeat 2 times. The EtOAc phase was then concentrated under reduced pressure to yield about 200 g of a yellowish clear product. The residue was taken into EtOH (2 L) and transferred to the separatory funnel. cHex (3 L) was added as well as water (100 mL). The phases were shaken vigorously, and the cHex phase was removed. Fresh cHex (1 L) was added, the phases were shaken vigorously, and the cHex phase was removed. The EtOH phase was concentrated under reduced pressure and the product was further dried overnight to yield about 160-170 g (75-80% yield) of a light yellowish clear product. MS (ESI): m/z=[M+H]⁺=573.5, 617.5 (100%), 661.6; [M+Ac]-=631.4, 675.4 (100%), 719.5. ¹H-NMR (600 MHz, CDCl₃): δ=7.33 (d, J=8.3 Hz, 2H), 7.23 (d, J=8.3 Hz, 2H), 4.52 (s, 2H), 3.72-3.58 (m, 33H), 2.54 (brs, 1H), 1.72 (s, 2H), 1.34 (s, 6H), 0.70 (s, 9H). ¹³C-NMR (150 MHz, CDCl₃): δ=149.7, 135.1, 127.4 (2C), 126.2 (2C), 73.2, 72.6, 70.7 (m), 70.5, 69.4, 61.9, 57.0, 38.6, 32.5, 31.9 (3C), 31.6 (2C). IR (cm⁻¹): 2957, 2865, 1465, 1350, 1249, 1097, 948, 816, 670, 632. IR (cm⁻¹): 2957, 2865, 1465, 1350, 1249, 1097, 948, 816, 670, 632. HRMS (ESI+) (m/z): [M+H]⁺ calculated for C₃₃H₆₁O₁₀ (n=9): 617.4265; found: 617.4269.

### 3.5 Synthesis of the Bi-Functional Side Product XVIII for Comparison

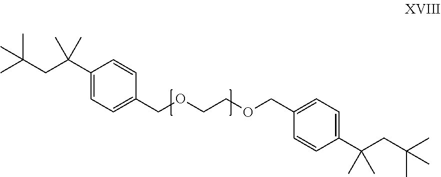

PEG400 (5.01 g, 12.5 mmol) was dissolved in THF (20 mL) at ambient temperature. tBuOK (3.25 g, 28.8 mmol) was added in one portion and the mixture was stirred 30 min. The benzyl bromide XVI was added to the deprotonated PEG400 solution at ambient temperature in one portion and the reaction was stirred overnight at ambient temperature. Water (120 g) and HCl (1 M, 10 mL) were added to the reaction mixture. The solution was transferred into a separatory funnel and EtOAc (120 mL) was added and extracted vigorously. After phase separation the organic phase was washed successively with water (2×100 mL) and finally concentrated after drying on MgSO₄ to yield 10.8 g of crude yellowish oily residue. This residue was charged on top of a SiO₂ column and eluted with CH₂Cl₂/MeOH (0 to 15%). Concentration of the pure fractions yielded 9.13 g (Yield: 91%) of a clear light yellowish oil. R_{f}=0.21-0.78 (CH₂Cl₂/MeOH 20:1). ¹H-NMR (600 MHz, CDCl₃): δ=7.33 (d, J=8.2 Hz, 4H), 7.23 (d, J=8.2 Hz, 4H), 4.53 (s, 4H), 3.80-3.49 (m, 36H), 1.73 (s, 4H), 1.35 (s, 12H), 0.71 (s, 18H). ¹³C-NMR (150 MHz, CDCl₃): δ=149.8 (2C), 135.2 (2C), 127.5 (4C), 126.3 (4C), 73.2, 70.8, 70.8 (m), 69.4, 57.1 (2C), 38.6 (2C), 32.5 (2C), 31.9 (6C), 31.7 (4C). IR (cm⁻¹): 2951, 2861, 1465, 1363, 1249, 1099, 817, 670, 633. HRMS (ESI+) (m/z): [M+Na]⁺ calculated for C₄₈H₈₂NaO₁₀ (n=9): 841.5806; found: 841.5797.

### Analysis Methods for the Analysis of Purity of the Final Product:

### HPLC Method 1:

The following settings were used for this method:
HPLC: Agilent 1260
Column: Zorbax Rx-C8, 5 μm, 4.6×250 mm (V=4.155 mL)
Eluents: water (A), acetonitrile (B)
Gradient elution: 70% B (0 min)→70% B (4 min)→97% B (20 min)→97% B (40 min)
Post time: 12 min
Flow: 1.0 mL/min
Column temperature: 25° C.
Injection volume: 5 μL
Wavelength: 200 nm

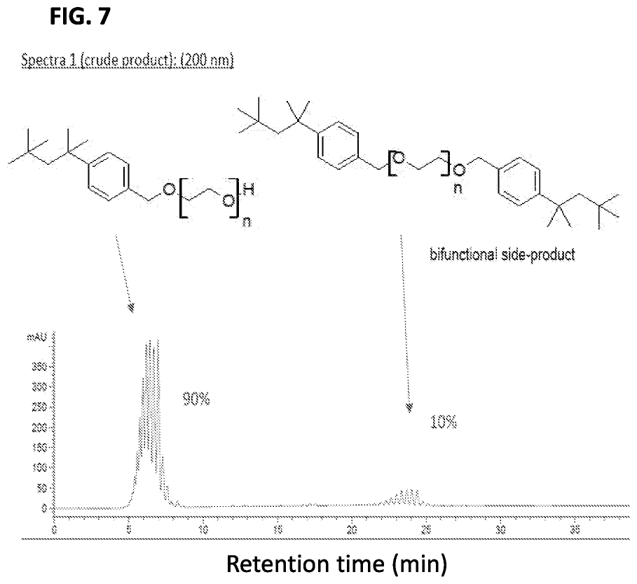

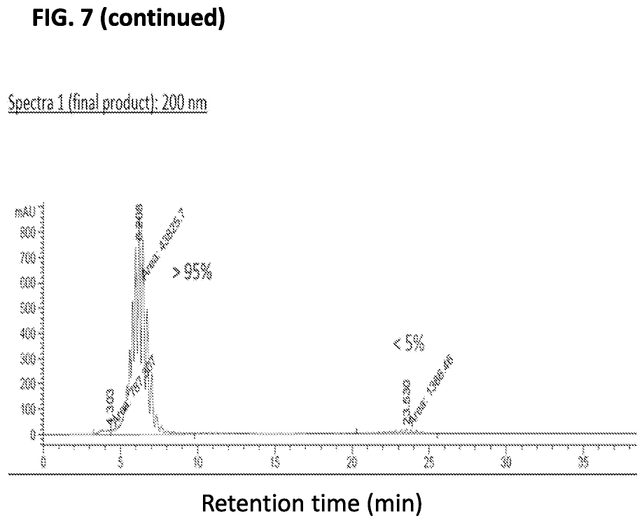

The spectra for the crude product at 200 nm and for the final product at 200 nm are shown in FIG. 7.

### HPLC Method 2:

The following settings were used for this method:
HPLC: Agilent 1260
Column: Zorbax 300SB-C3, 5 μm, 2.1×150 mm (V=0.520 mL)
Eluents: water (A), acetonitrile (B)
Gradient elution: gradient elution: 35% B (0 min)→35% B (4 min)→97% B (20 min)→97% B (2 5 min)
Post time: 8 min
Flow: 0.5 mL/min
Column temperature: 25° C.
Injection volume: 5 μL
Wavelength: 200 nm and ELSD

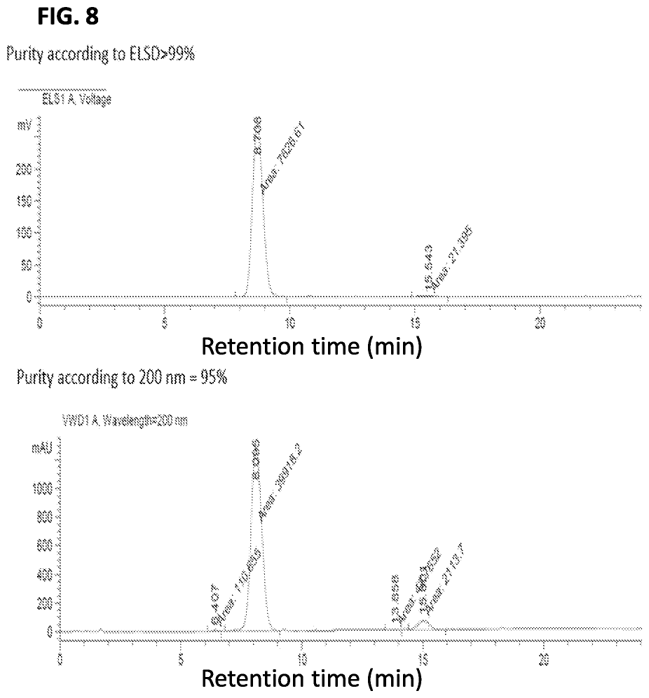

The spectra for the final product are shown in FIG. 8.

Purity according to ELSD>99%

Purity according to 200 nm=95%

### HPLC Method 3:

The following settings were used for this method:
HPLC: Agilent 1260
Column: Acclaim Surfactant Plus, 4.6×150 mm, 3 μm, 175 Å, V=2.493 mL
Eluents: water (A), acetonitrile (B)
Gradient elution: 20% B (0 min)→20% B (7 min)→90% B (27 min)→90% B (40 min)→20% B (40.1 min)→20% B (50 min)
Post time: 0 min
Flow: 0.5 mL/min
Column temperature: 30° C.
Injection volume: 5 μL
Wavelength: 225 nm
ELSD detection

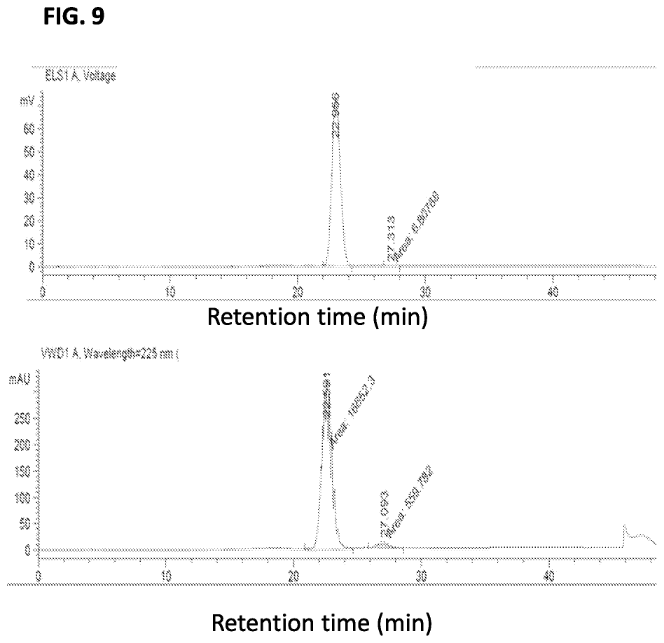

The spectra for the final product are shown in FIG. 9.

### Conclusions:

It can be noted that the route utilizes a low number of solvents (e.g., toluene, cHex, IPA, EtOAc and EtOH), all of them being non-hazardous and recoverable after concentration. A higher number of equivalents (eq.) of PEG, e.g., 5 molar equivalents (eq.) of PEG, used in the method are advantageous as compared to lower amounts of PEG, because they increase the purity of the product by reducing the amount of the bi-functional side product XVIII. If the addition of sodium hypochlorite is skipped the final product will have an orange coloration, the analytical results are however identical.

The 3-step, chromatography-free synthesis process described above uses inexpensive starting materials, has a robust and simple work-up, and allows production in a standard organic laboratory to deliver batches of several hundred grams with >99% purity. It can be used at an industrial scale for the preparation and purification of the detergents of the invention.

### Example 4: PRV Inactivation Using 4-tert-octylbenzyl Alcohol Polyethoxylate in Liquids Comprising Intravenous Immunoglobulin

### Materials and Methods

The experiments were performed as described for Example 1 of WO 2019/086463. However, for the S/D treatment an S/D mixture comprising the 4-tert-octylbenzyl alcohol polyethoxylate produced in Example 2a was tested and compared to an S/D mixture comprising Triton X-100. The composition of the S/D mixture comprising Triton X-100 was prepared as described in Example 1 of WO 2019/086463. The composition of the S/D mixture comprising 4-tert-octylbenzyl alcohol polyethoxylate was prepared as follows.

The S/D components Polysorbate 80 (PS80, Crillet 4 HP, Tween 80) and Tri-n-butyl-phosphate (TnBP) were combined with the detergent 4-tert-octylbenzyl alcohol polyethoxylate produced in Example 2a in the following ratios (see Table 1):

TABLE 1
Amount of components for 4-tert-octylbenzyl alcohol polyethoxylate
(abbreviated as “4-TOBAPE”) S/D Reagent Mix

| | Amount to be combined |
|---|---|
| S/D Reagent | for S/D Reagent Mix [g] |
| 4-TOBAPE | 10.61 ± 0.11 |
| PS80 | 3.23 ± 0.03 |
| TnBP | 2.93 ± 0.03 |
| Resulting S/D reagent | 16.77 |

The mixture was stirred for at least 15 minutes. The S/D reagent mix was stored at room temperature for use within one year. Prior to use the S/D reagent mix was stirred again for at least 15 minutes to assure homogeneity.

The respective S/D reagent mixes were added to the IVIG-containing liquid to give a final concentration of 0.05% w/w of the respective polyoxyethylene ether detergent.

Only inactivation of the virus PRV was tested.

### Results

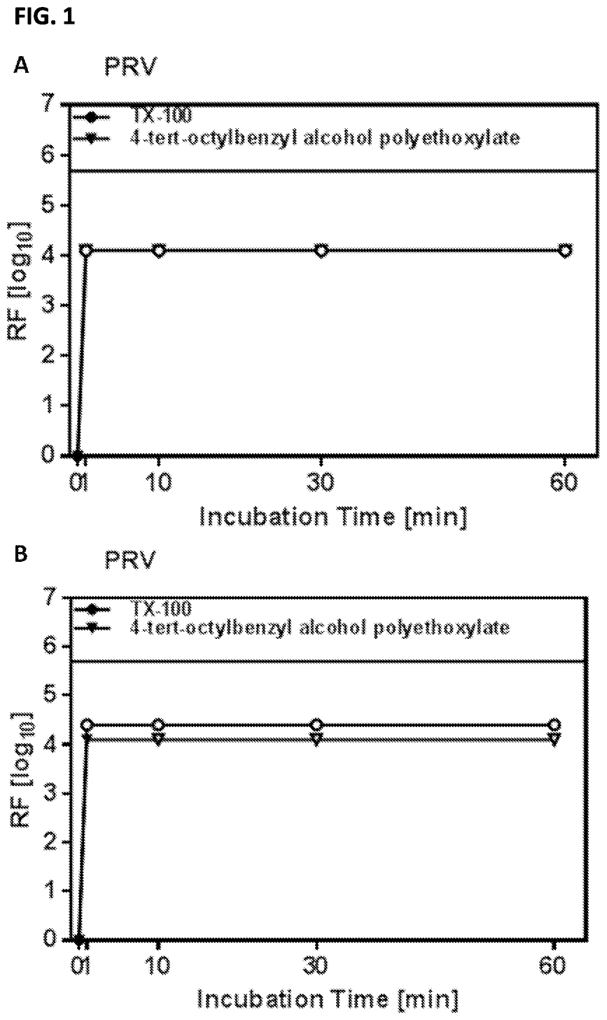

When S/D treatment of an IVIG-containing liquid was performed using a mixture of 4-tert-octylbenzyl alcohol polyethoxylate, PS80 and TnBP, PRV was inactivated by a virus reduction factor (RF) of around 4 within 1-2 minutes (FIG. 1A). Similar results were obtained in a repeat experiment (FIG. 1B).

These experiments show that replacing Triton X-100 by 4-tert-octylbenzyl alcohol polyethoxylate for S/D treatment of biopharmaceutical drug-containing liquids results in efficient inactivation of lipid-enveloped viruses, even at low concentrations of detergent.

### Example 5: X-MuLV Inactivation Using 4-tert-octylbenzyl Alcohol Polyethoxylate in Liquids Comprising Human Serum Albumin

### Materials and Methods

The experiments were performed as described for Example 3 of WO 2019/086463. However, for the S/D treatment an S/D mixture comprising 4-tert-octylbenzyl alcohol polyethoxylate was tested and compared to an S/D mixture comprising Triton X-100. The composition of the S/D mixture comprising Triton X-100 was prepared as described in Example 3 of WO 2019/086463. The composition of the S/D mixture comprising 4-tert-octylbenzyl alcohol polyethoxylate was prepared as follows.

The S/D components Polysorbate 80 (PS80, Crillet 4 HP, Tween 80) and Tri-n-butyl-phosphate (TnBP) were combined with the detergent 4-tert-octylbenzyl alcohol polyethoxylate in the following ratios (see Table 2):

TABLE 2
Amount of components for 4-tert-octylbenzyl alcohol polyethoxylate
(abbreviated as “4-TOBAPE”) S/D Reagent Mix

| | Amount to be combined |
|---|---|
| S/D Reagent | for S/D Reagent Mix [g] |
| 4-TOBAPE | 10.5 ± 0.1 |
| PS80 | 3.2 ± 0.03 |
| TnBP | 2.9 ± 0.03 |
| Resulting S/D reagent | 16.6 |

The mixture was stirred for at least 15 minutes. The S/D reagent mix was stored at room temperature for use within one year. Prior to use the S/D reagent mix was stirred again for at least 15 minutes to assure homogeneity.

The respective S/D reagent mixes were added to the HSA-containing liquid to give a final concentration of 0.1% of the respective polyoxyethylene ether detergent.

Only inactivation of the virus X-MuLV was tested.

### Results

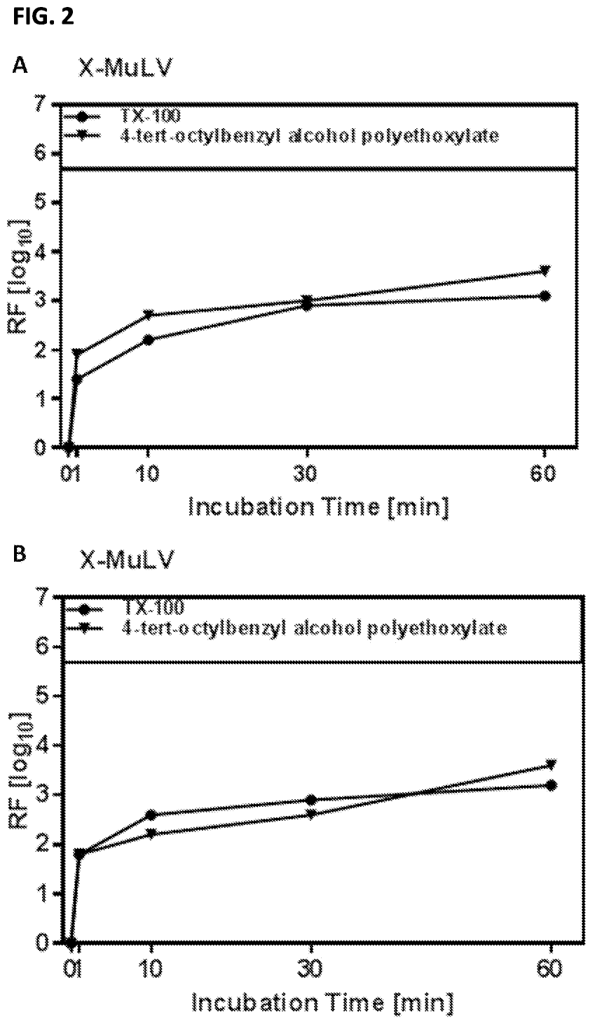

When S/D treatment of a HSA-containing liquid was performed at 1° C.±1C using a mixture of for 4-tert-octylbenzyl alcohol polyethoxylate, PS80 and TnBP, X-MuLV was inactivated by a virus reduction factor (RF) of over 2 within 10 minutes (FIG. 2A). Similar results were obtained in a repeat experiment (FIG. 2B).

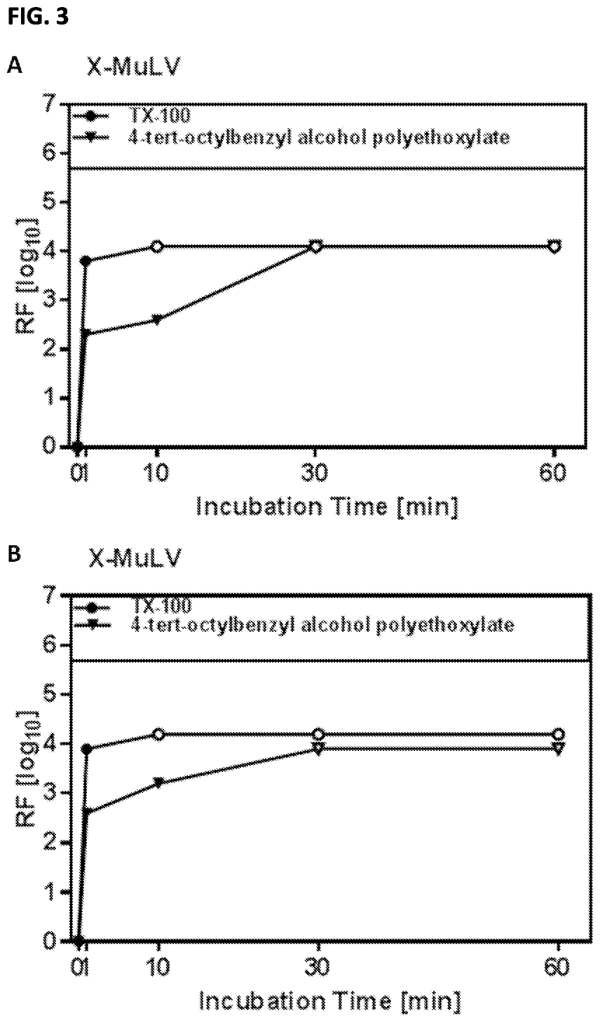

Additional experiments were performed exactly as described in the Materials and Methods section of this Example, only that S/D treatment of the HSA-containing liquid was performed at 19° C.±1° C. instead of 1° C.±1° C. When S/D treatment of a HSA-containing liquid was performed at 19° C.±1C using a mixture of for 4-tert-octylbenzyl alcohol polyethoxylate, PS80 and TnBP, X-MuLV was inactivated by a virus reduction factor (RF) of over 2 within 10 minutes (FIG. 3A), and by a RF of around 4 within 30 min. Similar results were obtained in a repeat experiment (FIG. 3B).

These experiments show that replacing Triton X-100 by for 4-tert-octylbenzyl alcohol polyethoxylate for S/D treatment of biopharmaceutical drug-containing liquids results in efficient inactivation of lipid-enveloped viruses at or around room temperature and at temperatures as low as 1° C.±1° C., even at low concentrations of the detergents.

### Example 6: BVDV Inactivation Using 4-tert-octylbenzyl Alcohol Polyethoxylate, Triton X-100 Reduced or Brij C10 in Buffer Containing HSA

The suitability of 4-tert-octylbenzyl alcohol polyethoxylate, Triton X-100 reduced or Brij C10 for single-detergent treatment to inactivate the lipid-enveloped virus bovine viral diarrhea virus (BVDV) in a buffer containing human serum albumin (HSA) as a model protein for a therapeutic antibody was tested and compared to Triton X-100. To this end, low concentrations of the respective detergent were added to a buffer containing HSA, and the mixture was then spiked with virus. Following incubation for various time periods the remaining infectivity of the virus was determined. The respective detergents were used at low concentrations in order to be able to evaluate the kinetics of virus inactivation, i.e., the efficiency of virus inactivation (expressed by the RF) over time. As will be clear to a person skilled in the art, in commercial production processes such as biopharmaceutical production, the detergents of the invention can be used at significantly higher concentrations, which will accelerate the kinetics of virus inactivation and may also increase the achieved RF.

### Materials

### Buffer Containing HSA

A buffer containing human serum albumin (HSA) was used as a model for a therapeutic antibody.

### Viruses

TABLE 3
Virus stock used in the experiments.

| Virus | | Propagated on | | Titrated on | |
|---|---|---|---|---|---|
| Strain | | Cell | | Cell | |
| BVDV | Source | line | Source | line | Source |
| Nadl | ATCC ¹ | MDBK | ATCC ¹ | BT | ATCC ¹ |
| | VR-1422 | | CRL-22 | | CRL-1390 |

¹ ATCC: American Type Culture Collection, 10801 University Boulevard, M [symbol] , VA 20110 USA [symbol]
indicates data missing or illegible when filed

Virus stocks were characterized prior to use. This characterization included the determination of the virus titer by at least ten independent titrations and specification of an acceptance virus titer range for use as positive control, the determination of virus stock protein content, PCR tests for virus identity and contamination with other viruses and mycoplasma and tests for virus aggregation with filters not allowing the passage of large virus aggregates. Only virus stocks passing PCR identity/contamination tests with no significant aggregation, (i.e. difference in infectivity titers between virus stock and filtered stock was smaller than 1.0 log) were used.

### Detergents

Prior to use the respective detergent, or a 1:10 dilution thereof (i.e. 1 g±2% of the respective detergent plus 9 g±2% of Aqua dest.), was stirred for at least 15 minutes to assure homogeneity.

### Methods

The virus inactivation capacity and the robustness of the single-detergent treatment were evaluated under conditions unfavorable for virus inactivation, i.e. with short incubation times and at relatively low temperatures. As will be clear to a person skilled in the art, in commercial production processes such as biopharmaceutical production. longer incubation times and higher temperatures can be used, which will accelerate the kinetics of virus inactivation and may also increase the achieved LRV. Further, as already mentioned above, low concentrations of the respective detergents were used. As will be clear to a person skilled in the art, in commercial production processes such as biopharmaceutical production, higher concentrations of the respective detergents can be used, which will accelerate the kinetics of virus inactivation and may also increase the achieved LRV.

Since the protein concentration has no significant impact on virus inactivation (see also Dichtelmüller et al., 2009), the robustness concerning protein content was not investigated.

All following steps took place in a biosafety class II cabinet. The starting material was incubated under stirring at a temperature of +14° C.±1° C. using a double-walled vessel connected to a cryostat for incubation.

The buffer containing HSA was transferred to a screw-cap flask of which the tare weight had been determined. The weight of the buffer containing HSA (in the closed flask) was determined for calculation of the amount of the single detergent to be added. The needed amount of detergent ([mg]), or the respective amount of a 1:10 dilution thereof, was added per g of buffer containing HSA to yield the following final concentrations of the respective detergent: 0.1%±0.01% (w/w) or 0.03%±0.01% (w/w). The detergent was added under stirring within 1 minute using a syringe, and the actual amount added was determined by back-weighing the syringe. The buffer containing HSA was stirred further after completed detergent addition for at least 10 minutes. The buffer containing HSA mixed with the detergent was filtered through a 0.2 μm Supor syringe membrane filter (or equivalent) and the filtrate was collected at a targeted temperature of 14° C.±1° C. using a double-walled vessel connected to a cryostat. The vessel for the filtrate was cooled. In case of filter clogging, fresh filters were used for continuing filtration of the buffer containing HSA. After filtration, the temperature (target: 14° C.±1C) and the volume were measured.

The filtered buffer containing HSA with the respective detergent was adjusted under stirring to 14° C.±1° C. using a double-walled vessel connected to a cryostat. This temperature range was maintained under stirring until the end of the incubation of the buffer containing HSA with the detergent and was recorded continuously. The determined volume of the buffer containing HSA with detergent was spiked at a ratio of 1:31, e.g. 48 mL buffer containing HSA were spiked with 1.6 ml of virus stock solution. The spiked buffer containing HSA was further incubated under continued stirring at 14° C.±1° C. for 59±1 minutes. During incubation, samples for virus titration were taken at 1-2 min, 5±1 min, 29±1 min and 59±1 min. To prevent further inactivation of virus by the detergent following sample drawing, the samples were diluted immediately 1:20 with the respective cold (+2° C. to +8° C.) cell culture medium (i.e. 1 volume of sample plus 19 volumes of cell culture medium).

Spike- and Hold Controls were performed on the same day: Buffer containing HSA was filtered as described above, but without prior addition of detergent. Then the filtrate was adjusted to 14° C.±1° C. under stirring as described above. The buffer containing HSA was spiked at a ratio of 1:31 and further incubated at 14° C.±1° C. for 59±1 minutes as described above. Within 1-2 minutes after spiking, a sample for virus titration (Spike Control) was taken. After incubation for 59±1 minutes a sample for virus titration (i.e. the Hold Control sample) was taken (HC).

Titration of the samples and calculation of the virus clearance capacity was performed as described for Example 1 of WO 2019/086463.

### Results

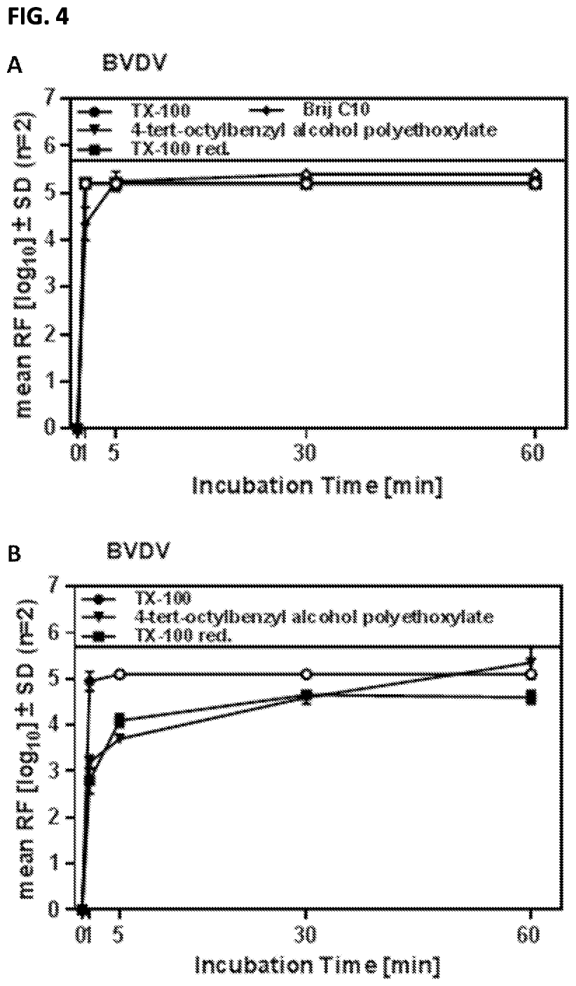

When single-detergent treatment of a buffer containing HSA was performed at 14° C.±1C using 0.1%±0.01% Triton X-100, Brij C10, 4-tert-octylbenzyl alcohol polyethoxylate or Triton X-100 reduced, BVDV was inactivated by a virus reduction factor (RF) of around 5 within 5 minutes (FIG. 4A). When single-detergent treatment of a buffer containing HSA was performed at 14° C.±1C using 0.03%±0.01% Triton X-100, 4-tert-octylbenzyl alcohol polyethoxylate or Triton X-100 reduced, BVDV was inactivated by a virus reduction factor (RF) of over 3 within 5 minutes, and of over 4 within 29 minutes (FIG. 4B).

These experiments indicate that single-detergent treatment of biopharmaceutical drug-containing liquids using Triton X-100, Brij C10, 4-tert-octylbenzyl alcohol polyethoxylate or Triton X-100 reduced results in efficient inactivation of lipid-enveloped viruses, even at low concentrations of the detergents.

### Example 7: BVDV Inactivation Using 4-tert-octylbenzyl Alcohol Polyethoxylate or Triton X-100 Reduced in IVIG-Containing Liquid

The suitability of 4-tert-octylbenzyl alcohol polyethoxylate or Triton X-100 reduced for single-detergent treatment to inactivate the lipid-enveloped virus bovine viral diarrhea virus (BVDV) in liquids comprising intravenous immunoglobulin (IVIG) was tested and compared to Triton X-100. To this end, virus was added to liquids comprising IVIG. The virus-containing liquids comprising IVIG were then incubated with low concentrations of Triton X-100 reduced, 4-tert-octylbenzyl alcohol polyethoxylate or Triton X-100 for various time periods, and the remaining infectivity of the viruses was determined. Triton X-100 reduced, 4-tert-octylbenzyl alcohol polyethoxylate and Triton X-100 were used at low concentrations in order to evaluate the kinetics of virus inactivation, i.e., the efficiency of virus inactivation (expressed by the RF) over time. As will be clear to a person skilled in the art, in commercial production processes such as biopharmaceutical production, the detergents of the invention including Triton X-100 reduced or 4-tert-octylbenzyl alcohol polyethoxylate can be used at significantly higher concentrations, which will accelerate the kinetics of virus inactivation and is also expected to increase the achieved LRV.

### Materials

### Liquid Comprising IVIG

A liquid comprising IVIG (IVIG-containing liquid) was frozen on dry ice and stored at ≤−60° C. until use within one year after the date of collection.

### Viruses

TABLE 4
Virus stock used in the experiments.

| Virus | | Propagated on | | Titrated on | |
|---|---|---|---|---|---|
| Strain | | Cell | | Cell | |
| BVDV | Source | line | Source | line | Source |
| Nadl | ATCC ¹ | MDBK | ATCC ¹ | BT | ATCC ¹ |
| | VR-1422 | | CRL-22 | | CRL-1390 |

¹ ATCC: American Type Culture Collection, 10801 University Boulevard, M [symbol] , VA 20110 USA [symbol]
indicates data missing or illegible when filed

Virus stocks were characterized prior to use. This characterization included the determination of the virus titer by at least ten independent titrations and specification of an acceptance virus titer range for use as positive control, the determination of virus stock protein content, PCR tests for virus identity and contamination with other viruses and mycoplasma and tests for virus aggregation with filters not allowing the passage of large virus aggregates. Only virus stocks passing PCR identity/contamination tests with no significant aggregation, (i.e. difference in infectivity titers between virus stock and filtered stock was smaller than 1.0 log) were used.

### Detergents

Prior to use the respective detergent, or a 1:10 dilution thereof (i.e. 1 g±2% of the respective detergent plus 9 g±2% of Aqua dest.), was stirred for at least 15 minutes to assure homogeneity.

### Methods

The virus inactivation capacity and the robustness of the single-detergent treatment were evaluated under conditions unfavorable for virus inactivation, i.e. with short incubation times and at relatively low temperatures. As will be clear to a person skilled in the art, in commercial production processes such as biopharmaceutical production, longer incubation times and higher temperatures can be used, which will accelerate the kinetics of virus inactivation and may also increase the achieved LRV. Further, as already mentioned above, low concentrations of the respective detergents were used. As will be clear to a person skilled in the art, in commercial production processes such as biopharmaceutical production, higher concentrations of the respective detergents can be used, which will accelerate the kinetics of virus inactivation and may also increase the achieved LRV.

Since the protein concentration has no significant impact on virus inactivation (see also Dichtelmüller et al., 2009), the robustness concerning protein content was not investigated.

The IVIG-containing liquid was thawed and all further steps took place in a biosafety class II cabinet. The IVIG-containing liquid was incubated under stirring at a temperature of +17° C.±1° C. using a double-walled vessel connected to a cryostat for incubation. The IVIG-containing liquid was then filtered through a 0.2 μm depth filter with an effective filter area of 25 cm² (Cuno VR06 or equivalent) connected to a Sartorius (SM16249) stainless steel filter holder using pressurized nitrogen at a targeted pressure of 0.9 bar (limit: 0.5 bar—1.5 bar). For conditioning the filter material was pre-coated with 55 L/m² of the Hyflo Supercel suspension (5.0 g±0.05 g of Hyflo Supercel per L; conductivity adjusted to 3.5 mS/cm (specified range: 2.5-6.0 mS/cm) using 3 M NaCl) (at a pressure of 0.5 bar) prior to filtration of the liquid. During filtration the filter holder was cooled and the filtrate was collected at a targeted temperature of +17° C.±1° C. using a double-walled vessel connected to a cryostat. The vessel for the filtrate was cooled. In case of filter clogging, fresh pre-conditioned filters were used for continuing filtration of the remaining liquid. After filtration the volume was measured.

After measuring the volume of the IVIG-containing liquid, it was adjusted under stirring with cold (+2° C. to +8° C.) dilution buffer (NaCl solution with target conductivity of 3.5 mS/cm (range: 2.5 mS/cm-6.0 mS/cm)) to a calculated target absorbance of 28.9 AU₂₈₀₋₃₂₀/cm (range: 14.5-72.3 AU₂₈₀₋₃₂₀/cm). Taking the later 1:31 virus spike into account, this resulted in a calculated target absorbance value of 28 AU₂₈₀₋₃₂₀/cm (range: 14-70 AU₂₈₀₋₃₂₀/cm) for incubation with the detergent after filtration.

The filtered and protein adjusted IVIG-containing liquid was again adjusted under stirring to +17° C.±1C using a double-walled vessel connected to a cryostat. This temperature range was maintained under stirring until the end of the incubation of the filtered IVIG-containing liquid with the detergent and was recorded continuously. The determined volume of the filtered IVIG-containing liquid, transferred to a screw-cap flask of which the tare weight had been determined, was spiked with virus at a ratio of 1:31, e.g. 30 mL of IVIG-containing liquid were spiked with 1 mL of virus stock solution. The spiked IVIG-containing liquid was further incubated under continued stirring at 17° C.±1° C. Within 1-2 minutes after spiking, samples for virus titration (Spike Control, SC, and Hold Control, HC) were taken.

After drawing, the Hold Control (HC) was kept at the same temperature, i.e. at +17° C. 1° C., as the spiked IVIG-containing liquid after addition of detergent, i.e. it was stored in the same cooling circle as the vessel with the IVIG-containing liquid, until the end of detergent treatment. The temperature of the cooling liquid was determined before insertion of the Hold Control sample and, again, shortly before the Hold Control was removed for titration after detergent treatment.

The weight of the spiked IVIG-containing liquid was determined for calculation of the amount of the detergent to be added. The weighed material was re-adjusted, if necessary, under stirring to +17° C.±1° C. The needed amount of detergent ([mg]), or the respective amount of a 1:10 dilution thereof, was added per g of IVIG-containing liquid to yield the following final concentrations of the respective detergent: 0.1%±0.01% (w/w) or 0.03%±0.01% (w/w). The detergent was added under stirring within 1 minute using a syringe, and the actual amount of detergent added was determined by back-weighing the syringe. The spiked IVIG-containing liquid was further incubated with the respective detergent under continued stirring at +17° C.±1C for 59±1 minutes. During incubation, 1 mL samples for virus titration were taken after 1-2 min, 10±1 min, 30±1 min and 59±1 min. To prevent further inactivation of virus by the S/D reagents following sample drawing, the samples were diluted immediately 1:20 with cold (+2° C. to +8° C.) cell culture medium (i.e. 1 volume of sample plus 19 volumes of cell culture medium).

Titration of the samples and calculation of the virus clearance capacity was performed as described for Example 1 of WO 2019/086463.

### Results

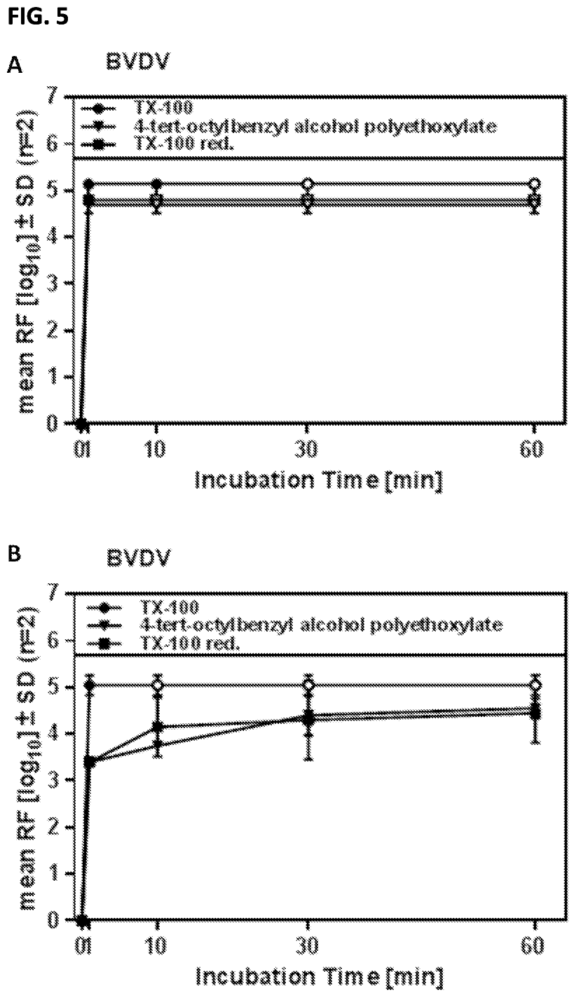

When single-detergent treatment of IVIG-containing liquid was performed at 17° C.±1C using 0.1%±0.01% Triton X-100, 4-tert-octylbenzyl alcohol polyethoxylate or Triton X-100 reduced, BVDV was inactivated by a virus reduction factor (RF) of around 5 within 10 minutes (FIG. 5A). When single-detergent treatment of IVIG-containing liquid was performed at 17° C.±1° C. using 0.03%±0.01% Triton X-100, 4-tert-octylbenzyl alcohol polyethoxylate or Triton X-100 reduced, BVDV was inactivated by a virus reduction factor (RF) of over 3 within 10 minutes, and of over 4 within 30 minutes (FIG. 5B).

These experiments confirm that single-detergent treatment of biopharmaceutical drug-containing liquids using Triton X-100, 4-tert-octylbenzyl alcohol polyethoxylate or Triton X-100 reduced results in efficient inactivation of lipid-enveloped viruses, even at low concentrations of the detergents.

### Example 8: BVDV Inactivation Using 4-tert-octylbenzyl Alcohol Polyethoxylate or Triton X-100 Reduced in FVIII-Containing Liquid

The suitability of 4-tert-octylbenzyl alcohol polyethoxylate or Triton X-100 reduced for single-detergent treatment to inactivate the lipid-enveloped virus bovine viral diarrhea virus (BVDV) in liquids comprising plasma-derived Factor VIII (pdFVIII) was tested and compared to Triton X-100. To this end, virus was added to liquids comprising pdFVIII. The virus-containing liquids comprising pdFVIII were then incubated with low concentrations of Triton X-100 reduced, 4-tert-octylbenzyl alcohol polyethoxylate or Triton X-100 for various time periods, and the remaining infectivity of the viruses was determined. Triton X-100 reduced, 4-tert-octylbenzyl alcohol polyethoxylate and Triton X-100 were used at low concentrations in order to evaluate the kinetics of virus inactivation, i.e., the efficiency of virus inactivation (expressed by the RF) over time. As will be clear to a person skilled in the art, in commercial production processes such as biopharmaceutical production, the detergents of the invention including Triton X-100 reduced or 4-tert-octylbenzyl alcohol polyethoxylate can be used at significantly higher concentrations, which will accelerate the kinetics of virus inactivation and is also expected to increase the achieved LRV.

### Materials

Liquid Comprising pdFVIII

A liquid comprising pdFVIII (pdFVIII-containing liquid) was frozen on dry ice and stored at ≤−60° C. until use within one year after the date of collection.

### Viruses

TABLE 5
Virus stock used in the experiments.

| Virus | | Propagated on | | Titrated on | |
|---|---|---|---|---|---|
| Strain | | Cell | | Cell | |
| BVDV | Source | line | Source | line | Source |
| Nadl | ATCC ¹ | MDBK | ATCC ¹ | BT | ATCC ¹ |
| | VR-1422 | | CRL-22 | | CRL-1390 |

¹ ATCC: American Type Culture Collection, 10801 University Boulevard, M [symbol] , VA 20110 USA [symbol]
indicates data missing or illegible when filed

Virus stocks were characterized prior to use. This characterization included the determination of the virus titer by at least ten independent titrations and specification of an acceptance virus titer range for use as positive control, the determination of virus stock protein content, PCR tests for virus identity and contamination with other viruses and mycoplasma and tests for virus aggregation with filters not allowing the passage of large virus aggregates. Only virus stocks passing PCR identity/contamination tests with no significant aggregation, (i.e. difference in infectivity titers between virus stock and filtered stock was smaller than 1.0 log) were used.

### Detergents

Prior to use the respective detergent, or a 1:10 dilution thereof (i.e. 1 g±2% of the respective detergent plus 9 g±2% of Aqua dest.), was stirred for at least 15 minutes to assure homogeneity.

### Methods

The virus inactivation capacity and the robustness of the single-detergent treatment were evaluated under conditions unfavorable for virus inactivation, i.e. with short incubation times. As will be clear to a person skilled in the art, in commercial production processes such as biopharmaceutical production, longer incubation times can be used, which will accelerate the kinetics of virus inactivation and may also increase the achieved LRV. Further, as already mentioned above, low concentrations of the respective detergents were used. As will be clear to a person skilled in the art, in commercial production processes such as biopharmaceutical production, higher concentrations of the respective detergents can be used, which will accelerate the kinetics of virus inactivation and may also increase the achieved LRV.

Since the protein concentration has no significant impact on virus inactivation (see also Dichtelmüller et al., 2009), the robustness concerning protein content was not investigated.

The pdFVIII-containing liquid was thawed and all further steps took place in a biosafety class II cabinet. To remove possible gross aggregates the pdFVIII-containing liquid was filtered through a 0.45 μm membrane filter (e.g. Sartorius SartoScale Sartobran or equivalent). The pdFVIII-containing liquid was transferred to a screw-cap flask of which the tare weight had been determined and adjusted under stirring to a targeted temperature of +23° C.±1° C. using a double-walled vessel connected to a cryostat. The determined volume of the pdFVIII-containing liquid was spiked at a ratio of 1:31, e.g. 48 mL of the pdFVIII-containing liquid were spiked with 1.6 mL of virus stock solution. Subsequently, a sample each for virus titration (Spike Control, SC) and for the Hold Control (HC) were taken.

The Hold Control was kept at the same temperature, i.e. at +23° C.±1C, as the spiked pdFVIII-containing liquid after addition of detergent, i.e. it was stored in the same cooling circle as the vessel with the pdFVIII-containing liquid, until the end of detergent treatment. The temperature of the cooling liquid was determined before insertion of the Hold Control sample and, again, shortly before the Hold Control was removed for titration after detergent treatment.

The weight of the spiked pdFVIII-containing liquid was determined for calculation of the amount of detergent to be added. The weighed material was adjusted under stirring to +23° C.±1° C. using a double-walled vessel connected to a cryostat. This temperature range was maintained under stirring until the end of the incubation of the spiked pdFVIII-containing liquid with the detergent and was recorded continuously. The needed amount of detergent ([mg]), or the respective amount of a 1:10 dilution thereof, was added per g of pdFVIII-containing liquid to yield a final detergent concentration of 0.1%±0.01% (w/w). The detergent was added under stirring within 1 minute using a syringe, and the actual amount of detergent added was determined by back-weighing the syringe. After the addition of the detergent had been completed, the spiked pdFVIII-containing liquid was further incubated under continued stirring at +23° C.±1° C. for 59±1 minutes. During incubation, 1 mL samples for virus titration were taken after 1-2 min. 5±1 min. 30±1 min and 59±1 min. To prevent further inactivation of virus by the detergent following sample drawing, the samples were diluted immediately 1:20 with the respective cold (+2 to +8° C.) cell culture medium (i.e. 1 volume of sample plus 19 volumes of cell culture medium).

Titration of the samples and calculation of the virus clearance capacity was performed as described for Example 1 of WO 2019/086463.

### Results

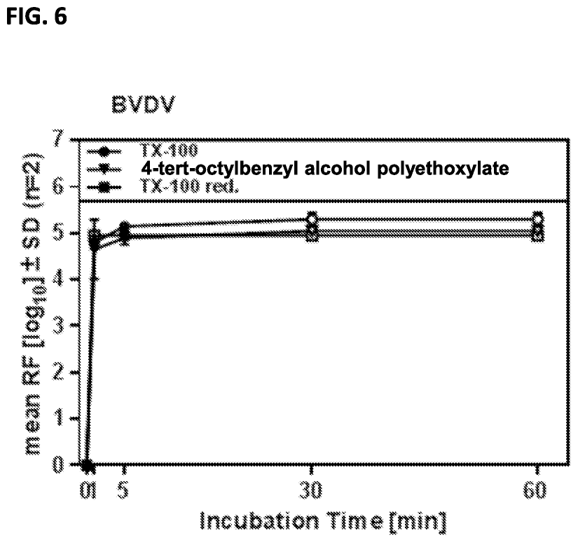

When single-detergent treatment of pdFVIII-containing liquid was performed at 23° C.±1C using 0.1%±0.01% Triton X-100, 4-tert-octylbenzyl alcohol polyethoxylate or Triton X-100 reduced, BVDV was inactivated by a virus reduction factor (RF) of around 5 within 5 minutes (FIG. 6).

These experiments confirm that single-detergent treatment of biopharmaceutical drug-containing liquids using Triton X-100, 4-tert-octylbenzyl alcohol polyethoxylate or Triton X-100 reduced results in efficient inactivation of lipid-enveloped viruses, even at low concentrations of the detergents.

### Example 9: Toxicology Tests

Based on in silico data, for none of the detergents according to the invention any evidence has been found to be active as endocrine disruptors.

### INDUSTRIAL APPLICABILITY

The methods and products of the invention are commercially useful, e.g. for environmentally compatible inactivation of lipid-enveloped viruses in industrial manufacturing processes. For example, the detergents obtainable by the methods of the invention can be used in the industrial production of biopharmaceuticals.

Thus, the invention is industrially applicable.

### REFERENCES

Dichtelmüller et al. (2009): Robustness of solvent/detergent treatment of plasma derivatives: a data collection from Plasma Protein Therapeutics Association member companies. Transfusion 49(9): 1931-1943.
Di Serio et al. (2005): Comparison of Different Reactor Types Used in the Manufacture of Ethoxylated, Propoxylated Products. Ind. Eng. Chem. Res. 44(25): 9482-9489.
ECHA, Support document for identification of 4-(1,1,3,3-tetramethylbutyl)phenol, ethoxylated as substances of very high concern because, due to their degradation to a substance of very high concern (4-(1,1,3,3-tetramethylbutyl)phenol) with endocrine disrupting properties, they cause probable serious effects to the environment which give rise to an equivalent level of concern to those of CMRs and PBTs/vPvBs, adopted on 12 Dec. 2012
Brochure “Global Assessment of the State-of-the-Science of Endocrine Disruptors” (WHO/PCS/EDC/02.2), published by the International Programme on Chemical Safety of the World Health Organization (2002)
Simons et al. (1973): Solubilization of the membrane proteins from Semliki Forest virus with Triton X100. J Mol Biol. 80(1):119-133.
U.S. Pat. No. 1,970,578
International Patent Publication No. WO 2019/086463
Vogel's Textbook of Practical Organic Chemistry ((5th Edition, 1989, A. I. Vogel, A. R. Tatchell, B. S. Furnis, A. J. Hannaford, P. W. G. Smith)
Bioorganic & Medicinal Chemistry, 16(9), 4883-4907; 2008: Effects of modifications of the linker in a series of phenylpropanoic acid derivatives: Synthesis, evaluation as PPARα/γ dual agonists, and X-ray crystallographic studies; Casimiro-Garcia, Agustin; Bigge, Christopher F.; Davis, Jo Ann; Padalino, Teresa; Pulaski, James; Ohren, Jeffrey F.; McConnell, Patrick; Kane, Christopher D.; Royer, Lori J.; Stevens, Kimberly A.; Auerbach, Bruce J.; Collard, Wendy T.; McGregor, Christine; Fakhoury, Stephen A.; Schaum, Robert P.; Zhou, Hairong. DOI:10.1016/j.bmc.2008.03.
Chemistry—A European Journal, 23(60), 15133-15142; 2017: Solid Phase Stepwise Synthesis of Polyethylene Glycols; Khanal, Ashok; Fang, Shiyue.DOI:10.1002/chem.201703004
Journal of Medicinal Chemistry, 48(10), 3586-3604; 2005: Synthesis and Structure-Activity Relationships of Novel Selective Factor Xa Inhibitors with a Tetrahydroisoquinoline Ring; Ueno, Hiroshi; Yokota, Katsuyuki; Hoshi, Jun-Ichi; Yasue, Katsutaka; Hayashi, Mikio; Hase, Yasunori; Uchida, Itsuo; Aisaka, Kazuo; Katoh, Susumu; Cho, Hidetsura. DOI:10.1021/jm058160e
Journal of Nanoparticle Research, 15(11), 2025/1-2025/12, 12 pp.; 2013: Magnetic nanoparticles conjugated to chiral imidazolidinone as recoverable catalyst; Mondini, Sara; Puglisi, Alessandra; Benaglia, Maurizio; Ramella, Daniela; Drago, Carmelo; Ferretti, Anna M.; Ponti, Alessandro. DOI:10.1007/s11051-013-2025-3
Journal of Organic Chemistry, 79(1), 223-229; 2014: A Scalable Procedure for Light-Induced Benzylic Brominations in Continuous Flow; Cantillo, David; de Frutos, Oscar; Rincon, Juan A.; Mateos, Carlos; Kappe, C. Oliver. DOI:10.1021/jo402409k
Journal of Physical Chemistry B, 107(31), 7896-7902; 2003: Anellated hemicyanine dyes with large symmetrical solvatochromism of absorption and fluorescence; Huebener, Gerd; Lambacher, Armin; Fromherz, Peter. DOI:10.1021/jp0345809
PCT Int. Appl., 2005016240, 24 Feb. 2005: Preparation of aryl carbamate oligomers for hydrolyzable prodrugs and prodrugs comprising same; Ekwuribe, Nnochiri N.; Odenbaugh, Amy L. WO 2004-US15004, May 6, 2004
Russian Journal of Applied Chemistry, 82(6), 1029-1032; 2009: Relative activity of alkenyl-gem-dichlorocyclopropanes in the reactions of hydrogenation and alkylation; Brusentsova, E. A.; Zlotskii, S. S.; Kutepov, B. I.; Khazipova, A. N. DOI:10.1134/S1070427209060196
Russian Journal of Organic Chemistry, 51(11), 1545-1550; 2015: Alkylation of aromatic compounds with 1-bromoadamantane in the presence of metal complex catalysts; Khusnutdinov, R. I.; Shchadneva, N. A.; Khisamova, L. F.

1. A method for preparing and purifying a compound of the following general Formula (XIX) 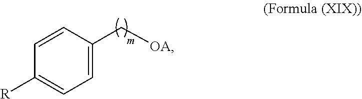
wherein m=1, R represents a hydrocarbon group having a linear chain of 2 to 12 carbon atoms and one or more methyl groups as substituents on said linear chain, and A represents a polyoxyethylene residue,
the method comprising the steps of
(3) Reacting a compound of the following general Formula (XII) 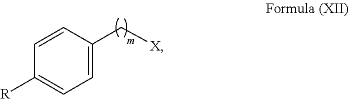
wherein m and R are as defined above and X represents a halogen atom or a hydroxyl group,
with a polyethylene glycol (PEG) of the general formula HO(CH₂CH₂O)ₙH, wherein n is an integer of n≥2, to produce a compound of said general formula (XIX) and a side product of the following general Formula (XIII) 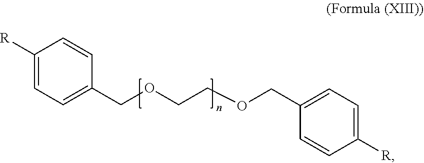
wherein R is as defined above and n is an integer of n≥2;
and
(4) Purifying the compound of said general Formula (XIX) by removing therefrom unreacted polyethylene glycol (PEG) and said side product.

2. The method of claim 1, which further comprises the following step (2) prior to said step (3):
(2) Converting a compound of the following general Formula (XIV), 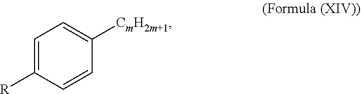
wherein m and R are as defined in claim 1,
into a compound of the following general Formula (XII) 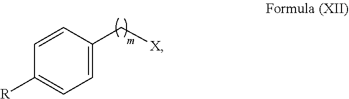
wherein m, R and X are as defined in claim 1.

3. The method of claim 2, which further comprises the following step (1) prior to said step (2):
(1) Reacting toluene so as to obtain a compound of the following general Formula (XIV) 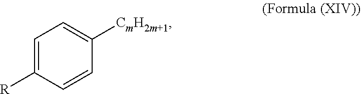
wherein m and R are as defined in claim 1.

4. The method of any one of the preceding claims, wherein in step (3), said polyethylene glycol (PEG) is used both as a reactant and as a solvent.

5. The method of claim 3 or 4, wherein in step (1), toluene is reacted with diisobutylene or a compound R-X, wherein R is as defined in claim 1 and X represents a halogen atom.

6. The method of any of claims 2 to 5, wherein the converting in step (2) is a radical reaction using AIBN (azobis(isobutyronitrile)) as a radical initiator.

7. The method of any of claims 1 to 6, wherein X is a chlorine, iodine or bromine atom.

8. The method of any of claims 1 to 7, wherein X is a bromine atom.

9. The method of any of claims 2 to 8, wherein the converting in step (2) uses N-bromosuccinimide (NBS) as a halogenating reagent.

10. The method of any one of the preceding claims, wherein said step (4) comprises subjecting a solution of the compound of said general Formula (XIX) in a solvent selected from alkanes, ethers and esters and mixtures thereof to one or more aqueous washes to remove said unreacted polyethylene glycol (PEG) from the compound of said general Formula (XIX).

11. The method of claim 10, wherein said solvent selected from alkanes, ethers and esters and mixtures thereof is an ester.

12. The method of claim 11, wherein said solvent selected from alkanes, ethers and esters and mixtures thereof is ethyl acetate.

13. The method of any one of the preceding claims, wherein the purifying of said compound of said general Formula (XIX) by removing therefrom said side product in step (4) comprises subjecting a solution of said compound of said general Formula (XIX) in a mixture comprising water and a water-miscible organic solvent to one or more washes with a non-polar solvent.

14. The method of claim 13, wherein the non-polar solvent comprises an alkane.

15. The method of claim 14, wherein the alkane is hexane, cyclohexane or heptane.

16. The method of claim 15, wherein the alkane is cyclohexane.

17. The method of any one of the preceding claims, wherein step (3) is performed without employing a solvent or diluent other than said polyethylene glycol (PEG).

18. The method of any one of the preceding claims, wherein no halogenated solvent is used.

19. The method of any one of the preceding claims, which does not include the use of any preparative chromatographic methods after step (3).

20. The method of any one of the preceding claims, which does not include the use of any preparative chromatographic methods.

21. The method of any one of the preceding claims, wherein the reacting in step (3) takes place for at least 10 minutes and for not more than 1 hour.

22. The method of any one of the preceding claims, wherein the reacting in step (3) takes place for at least 20 minutes and for not more than 45 minutes.

23. The method of any one of the preceding claims, wherein the reacting in step (3) takes place for 30 minutes.

24. The method of any one of the preceding claims, wherein the reacting in step (3) takes place at a temperature of between 55° C. and 65° C.

25. The method of any one of the preceding claims, wherein the reacting in step (3) takes place at a temperature of 60° C.

26. The method of any one of the preceding claims, wherein in step (3), each molar equivalent (eq.) of said compound of said general Formula (XII) is reacted with at least 1.0, at least 2.0, at least 3.0 or at least 4.0 molar equivalents (eq.) of said polyethylene glycol (PEG).

27. The method of any one of the preceding claims, wherein in step (3), each molar equivalent (eq.) of said compound of said general Formula (XII) is reacted with at least 4.5 molar equivalents (eq.) of said polyethylene glycol (PEG).

28. The method of any one of the preceding claims, wherein in step (3), each molar equivalent (eq.) of said compound of said general Formula (XII) is reacted with at least 5.0 molar equivalents (eq.) of said polyethylene glycol (PEG).

29. The method of any one of the preceding claims, wherein said polyethylene glycol (PEG) used in step (3) is characterized in that it is liquid at the reaction temperature of said step (3).

30. The method of any one of the preceding claims, wherein the method is carried out at a scale which yields at least 100 g, at least 1 kg, at least 10 kg, at least 100 kg or at least 1000 kg of said compound of said Formula (XIX).

31. The method of any one of claims 2-30, wherein the compound of formula (XII) obtained in step (2) is precipitated from a solvent.

32. The method of claim 31, wherein the solvent is an alcohol solvent.

33. The method of any one of claims 31-32, wherein the solvent is a polar solvent.

34. The method of any one of claims 32-33, wherein the alcohol solvent is selected from the group consisting of isopropyl alcohol, ethanol, methanol and butanol or combinations thereof.

35. The method of any one of claims 32-34, wherein the alcohol solvent is a secondary alcohol.

36. The method of any one of claims 32-35, wherein the alcohol solvent is isopropyl alcohol.

37. The method of any one of claims 32-33, wherein the solvent is acetonitrile.

38. The method of any one of claims 2-37, wherein at least one recrystallization of said compound of Formula (XII) is carried out in between step (2) and step (3).

39. The method of claim 38, wherein the solvent of any one of claims 31 to 37 is employed as solvent for the recrystallization between step 2 and step 3.

40. The method of any one of the preceding claims, wherein at least 1, at least 2, at least 3 or at least 4 aqueous washes are carried out after step 3.

41. The method of any one of the preceding claims, wherein 4 to 6 aqueous washes are carried out after step 3.

42. A method of purifying a compound of formula (XIX), 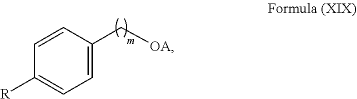
wherein m=1, R represents a hydrocarbon group having a linear chain of 2 to 12 carbon atoms and one or more methyl groups as substituents on said linear chain, and A represents a polyoxyethylene residue,
from a composition comprising the compound of formula (XIX), the method comprising subjecting a solution of said composition in a solvent selected from alkanes, ethers and esters and mixtures thereof to one or more aqueous washes.

43. The method of claim 42, wherein said solvent selected from alkanes, ethers and esters and mixtures thereof is an ester.

44. The method of claim 43, wherein said solvent selected from alkanes, ethers and esters and mixtures thereof is ethyl acetate.

45. The method of any of claims 42 to 44, wherein the composition comprises polyethylene glycol (PEG).

46. The method of claim 45, wherein the purifying of the compound of formula (XIX) comprises removing the polyethylene glycol (PEG) from the composition.

47. The method of claim 46, wherein the removing of the polyethylene glycol (PEG) comprises said subjecting of the solution to the one or more aqueous washes.

48. The method of any one of claims 42 to 47, wherein the composition further comprises a compound of the following general Formula (XIII): 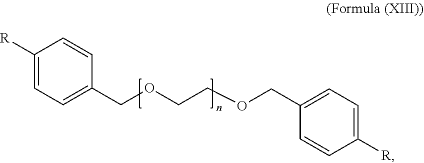
and wherein R is as defined in claim 42 and n is an integer of n≥2.

49. The method of claim 48, wherein the purifying of the compound of formula (XIX) comprises removing the compound of formula (XIII) from the composition.

50. The method of claim 49, wherein the removing of the compound of formula (XIII) comprises preparing an aqueous solution comprising said compound of said general Formula (XIX) and removing said compound of formula (XIII) by one or more washes of said aqueous solution with a non-polar solvent.

51. The method of claim 50, wherein the non-polar solvent comprises an alkane.

52. The method of claim 51, wherein the alkane is hexane, cyclohexane or heptane.

53. The method of claim 52, wherein the alkane is cyclohexane.

54. The method of any one of claims 1 to 53, wherein the degree of purity of the compound of formula (XIX) as obtained by the method, as measured by high-performance liquid chromatography with UV detection at 200 nm, is at least 80%, preferably at least 85%, more preferably at least 90%, and most preferably at least 95%.

55. The method of any one of claims 1 to 54, wherein the degree of purity of the compound of formula (XIX) as obtained by the method, as measured by high-performance liquid chromatography with detection using an evaporative light scattering detector (ELSD), is at least 99%.

56. The method of any one of the preceding claims, further comprising a step of identifying a compound of the following general Formula (XIII): 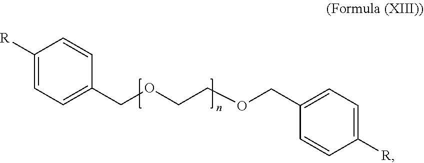
wherein R represents a hydrocarbon group having a linear chain of 2 to 12 carbon atoms and one or more methyl groups as substituents on said linear chain, and n is an integer of n≥2.

57. The method of any one of the preceding claims, further comprising a step of quantifying a compound of the following general Formula (XIII): 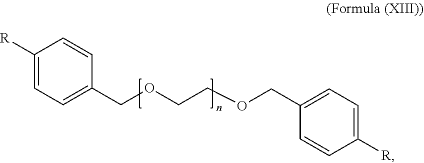
wherein R represents a hydrocarbon group having a linear chain of 2 to 12 carbon atoms and one or more methyl groups as substituents on said linear chain, and n is an integer of n≥2.

58. The method of any one of the preceding claims, wherein A represents a polyoxyethylene residue comprising 4 to 16 oxyethylene units.

59. The method of any one of the preceding claims, wherein A represents a polyoxyethylene residue comprising 8 to 10 oxyethylene units.

60. The method of any one of the preceding claims, wherein A represents a polyoxyethylene residue comprising 9 or 10 oxyethylene units.

61. The method of any one of the preceding claims, wherein R represents a hydrocarbon group having a linear chain of 2 to 6 carbon atoms and one or more methyl groups as substituents on said linear chain.

62. The method of any one of the preceding claims, wherein R represents a hydrocarbon group having a linear chain of 2 to 6 carbon atoms and 2 to 4 methyl groups as substituents on said linear chain.

63. The method of any one of the preceding claims, wherein R represents a hydrocarbon group having a linear chain of 4 carbon atoms and 4 methyl groups as substituents on said linear chain.

64. The method of any one of the preceding claims, wherein R represents a 2,4,4-trimethyl-pent-2-yl group.

65. The method of any one of the preceding claims, wherein the compound of Formula (XIX) is the following compound: 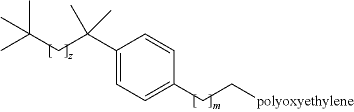
wherein m equals 1 and z is an integer selected from:
z=1 to 5.

66. The method of any one of the preceding claims, wherein the compound of Formula (XIX) is the following compound: 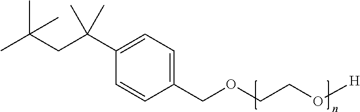
wherein n is an integer between 4 and 16, preferably wherein n is equal to 8, 9 or 10.

67. A compound of the following general Formula (XIX) 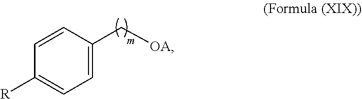
wherein m=1, R represents a hydrocarbon group having a linear chain of 2 to 12 carbon atoms and one or more methyl groups as substituents on said linear chain, and A represents a polyoxyethylene residue,
and wherein said compound is obtainable by the method of any one of the preceding claims.

68. The compound of claim 67, which exhibits a degree of purity, as measured by high-performance liquid chromatography with UV detection at 200 nm, of at least 80%, preferably at least 85%, more preferably at least 90%, and most preferably at least 95%.

69. The compound of claim 67 or 68, which exhibits a degree of purity, as measured by high-performance liquid chromatography with detection using an evaporative light scattering detector (ELSD), of at least 99%.

70. Use of a non-polar solvent for extracting and removing a compound of the following general Formula (XIII): 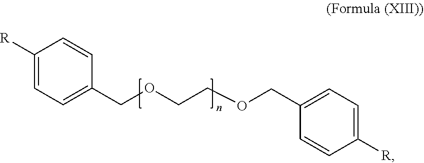
wherein R represents a hydrocarbon group having a linear chain of 2 to 12 carbon atoms and one or more methyl groups as substituents on said linear chain and n is an integer of n≥2,
from a composition comprising said compound of Formula (XIII) and a compound of formula (XIX), 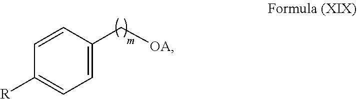
wherein m=1, R represents a hydrocarbon group having a linear chain of 2 to 12 carbon atoms and one or more methyl groups as substituents on said linear chain, and A represents a polyoxyethylene residue.

71. The use of claim 70, wherein the non-polar solvent is as defined in any one of claims 51-53.

72. The use of claim 70 or 71, wherein A is as defined in any one of claims 58-60.

73. The use of any one of claims 70 to 72, wherein R is as defined in any one of claims 61-64.

74. The use of any one of claims 70 to 73, wherein the compound of Formula (XIX) is as defined in any one of claims 65-66.
